(19) United States
(12) Patent Application Publication
(10) Pub. No.: US 2002/0034829 A1
(43) Pub. Date: Mar. 21, 2002

(54) Combinatorial synthesis and analysis of metal-ligand compositions using soluble metal precursors

(76) Inventors: Keith Hall, San Jose, CA (US); Vince Murphy, Campbell, CA (US); Anne M. LaPointe, Sunnyvale, CA (US); Johannes A.M. van Beek, Brussels (BE); Gary M. Diamond, San Jose, CA (US)

Correspondence Address: SENNIGER POWERS LEAVITT AND ROEDEL, ONE METROPOLITAN SQUARE, 16TH FLOOR, ST LOUIS, MO, 63102, US

(21) Appl. No.: 09/817,821

(22) Filed: Mar. 26, 2001

Related U.S. Application Data
(60) Provisional application No. 60/191,933, filed on Mar. 24, 2000.

(51) Int. Cl. G01N 33/543; C08F 4/06

(52) U.S. Cl. 436/518; 526/90

(57) ABSTRACT

The present invention relates to a process of conducting research using combinatorial techniques wherein metal-ligand compositions are synthesized and screened for reactivity in reactions of interest, particularly metal-ligand compounds which are suitable for use as catalysts. In the process, an array of different metal-ligand compositions is prepared by delivering a dissolved, soluble metal precursor and a metal-binding ligand to each reaction vessel or well in the array, where they may optionally be combined with an activator and/or other additives. The resulting metal-ligand composition in each vessel is then screened for reactivity in a chemical reaction of interest, particularly for catalytic activity in a polymerization reaction.

### BACKGROUND OF THE INVENTION

The present invention relates to a process of conducting research using combinatorial techniques wherein metal-ligand compositions are synthesized and screened for reactivity in reactions of interest, particularly metal-ligand compounds which are suitable for use as catalysts. More specifically, this invention is directed to a process wherein solutions comprising dissolved, soluble metal precursors are delivered to an array of reaction wells, where they may optionally be combined with ligands, activators, and/or other additives, the contents of one or more of these reaction wells then being screened for reactivity in a chemical reaction of interest, particularly catalytic activity in polymerization reactions.

Combinatorial chemistry has revolutionized the process of drug discovery. (See, for example, 29 Acc. Chem. Res. 1-170 (1996); 97 Chem. Rev. 349-509 (1997); S. Borman, Chem. Eng. News 43-62 (Feb. 24, 1997); A. M. Thayer, Chem. Eng. News 57-64 (Feb. 12, 1996); and, N. Terret, 1 Drug Discovery Today 402 (1996).) Because of its success in eliminating the synthesis bottleneck in drug discovery, many researchers have come to narrowly view combinatorial methods as tools for creating structural diversity. Few researchers have emphasized that, during synthesis, variations in temperature, pressure, ionic strength and other process conditions can strongly influence the resulting properties of library members. For example, reaction conditions are particularly important in formulation chemistry and polymer chemistry, where one combines a set of components under different reaction conditions or concentrations to determine their influence on product properties.

Recently, combinatorial approaches have been used for discovery programs unrelated to drugs. Combinatorial materials science generally refers to the methods for creating a collection of chemically diverse compounds or materials, and to methods for rapidly testing or screening this library of compounds or materials for desirable performance characteristics and properties. For example, some researchers have recognized that combinatorial strategies offer promise for the discovery of inorganic compounds such as high-temperature superconductors, magnetoresistive materials, luminescent materials and catalytic materials. (See, for example, U.S. Pat. No. 5,985,715 entitled “The Combinatorial Synthesis of Novel Materials” and U.S. Pat. No. 5,776,359, which are both herein incorporated by reference.) Compared to traditional discovery methods, combinatorial methods sharply reduce the costs associated with preparing and screening each candidate material.

Some combinatorial research into catalysis and polymer formation has begun. (See, for example, U.S. Pat. No. 6,030,917 entitled “Combinatorial Synthesis and Analysis of Organometallic Compounds and Catalysts,” as well as the following articles, which discuss one or more combinatorial techniques in conjunction with catalysis and which are incorporated herein by reference: Senkan, Nature, vol 394, pp. 350-353 (Jul. 23, 1998); Burgess et al., Angew. Chem. Int. Ed. Eng., 1996, 35, No. 2, pp. 220-222; Maier et al., Angew. Chem. Int. Ed. Eng., 1998, 37, No. 19, pp. 2644-2647; Reetz et al., Angew. Chem. Int. Ed. Eng., 1998, 37, No. 19, pp. 2647-2650; Schlögl, Angew. Chem. Int. Ed. Eng., 1998, 37, No. 17, pp. 2333-2336; Morken et al., Science, vol. 280, pp. 267-270 (Apr. 10, 1998); and, Gilbertson et al., Tetrahedron Letters, vol. 37, no. 36, pp. 6475-6478 (1996).) In these combinatorial approaches, arrays of potentially active catalytic compounds are synthesized in a parallel or rapid serial fashion, usually on a scale that produces only the amount of catalyst needed for purposes of screening it in a particular catalytic transformation. Accordingly, the amount of catalyst generated in such a synthesis is typically many orders of magnitude smaller than that produced by conventional means (wherein millimolar, or greater, amounts of a single catalytic compound are produced).

In order to achieve an efficient, high yield synthesis of a catalyst target on such a small scale, it is important to be able to carefully control the reactant stoichiometry (i.e., the ratio of metal precursor to ligand(s)), as well as the order, temperature and rate of addition of the reactants. Furthermore, the preparation of such arrays of catalysts is most advantageously performed using an automated transfer of reagents to the reaction wells in the array, including the metal precursors which are to be combined with one or more ligands, and with a minimum number of synthetic steps and isolation/purification protocols.

One approach to achieving these and other results is by means of a solution-based process. Ideally, such a process would employ the use of a soluble metal precursor dissolved in a suitable solvent. This approach would act to address the challenges of accurately measuring and transferring very small quantities of metal precursors, and would help to minimize process steps (by potentially eliminating, for example, isolation/purification steps that might otherwise be needed).

### SUMMARY OF THE INVENTION

Among the several features of the present invention, thereof, may be noted the provision of a process for preparing an array of metal-ligand compositions for screening in a reaction of interest; the provision of such a process wherein the compositions are formed by combining member ligands from a ligand array with a dissolved, soluble metal precursor; the provision of such a process wherein the resulting metal-ligand compositions are screened for catalytic activity; the provision of such a process wherein the metal-ligand compositions are screened for catalytic activity in polymerization reactions; the provision of such a process wherein the metal-ligand compositions are screened for catalytic activity in olefin polymerization reactions; the provision of such a process wherein the soluble metal precursor comprises a metal and at least one solublizing group, a solublizing group being displaced as a result of metal-ligand composition formation; and, the provision of such a process wherein the displaced solublizing group does not interfere with the reaction of interest.

Further among the several features of the present invention may be noted the provision of a metal precursor, suitable for use in the present process, which is capable of being dissolved in a suitable solvent, the metal precursor having a central metal atom and one or more solublizing ligands attached thereto.

Briefly, therefore, the present invention is directed to a process for preparing and screening an array of metal-ligand compositions. The process comprises: (i) preparing an array of polymerization mixtures in a series of discrete reaction vessels contained by or within an integrated structure, each polymerization mixture of the array comprising a polymerization monomer and a metal-ligand composition wherein different reaction vessels of the array contain different metal-ligand compositions and said preparing comprises delivering a metal-binding ligand and a dissolved, soluble metal precursor to each of the reaction vessels of the array which combine to form the metal-ligand composition; (ii) subjecting the integrated structure to conditions conducive to the formation of a polymerization reaction product; and, (iii) screening the array for polymerization reaction product.

The present invention is further directed to a process for preparing and screening an array of metal-ligand compositions. The process comprises: (i) preparing an array of reaction mixtures in a series of discrete reaction vessels contained by or within an integrated structure, each reaction mixture of the array comprising, in a non-protic medium, a reactant for a chemical reaction of interest and a metal-ligand composition for catalyzing a chemical reaction in which the reactant participates wherein different reaction vessels of the array contain different metal-ligand compositions and said preparing comprises delivering a metal-binding ligand not bound to a solid material and a dissolved, soluble metal precursor to each of the reaction vessels of the array; (ii) subjecting the reaction mixture to conditions conducive to the formation of a reaction product; and (iii) screening the array for reaction product.

Other features of the present invention will be in part apparent and in part pointed out hereinafter.

### DETAILED DESCRIPTION OF THE PREFERRED EMBODIMENTS

In accordance with the present invention, a combinatorial process has been discovered wherein an array or library of metal-ligand compositions, and in some embodiments compounds or complexes, are: (i) prepared by means of combining a plurality of member ligands from a ligand array with one or more solutions comprising a dissolved, soluble metal precursor at different sites on a substrate (e.g., multiple reaction wells or vessels contained by or within an integrated structure), and then (ii) screened for reactivity in one or more chemical reactions of interest. The present process is therefore advantageous in that, because the metal is in a solublized form, automated solution handling devices common in the art may be used, which in turn enables the efficient preparation of a library comprising a plurality of metal-ligand compositions (i.e, about 8, 20, 50, 80, 100, 300, 500, 1000, 10000, 106 or more) . Additionally, the use of a dissolved, soluble metal precursor enables these metal-ligand compositions to be prepared such that: (i) a by-product of the reaction between the soluble metal precursor and a ligand is not formed (e.g., a solublizing ligand is not displaced from the soluble metal precursor); or, (ii) an innocuous by-product of this reaction is formed (i.e., a by-product which either (a) does not interfere with or significantly inhibit a subsequent screening reaction, or (b) can be easily removed or treated in a way which renders it unable to interfere with or significantly inhibit such a reaction).

In this regard it is to be noted that, as typically used herein, “soluble” metal precursor generally refers to a metal-ligand complex, composition or compound which is capable of forming at least about a 1 mM (millimolar) solution, 25 mM solution, 50 mM solution, 75 mM solution, 100 mM solution or more, in an appropriate solvent (preferably in an aprotic solvent, and more preferant in a nonpolar, aprotic solvent). Additionally, as typically used herein, a “displaced” solublizing ligand generally refers to a ligand which has been separated from the metal(s) of the soluble metal precursor as a result of breaking of the covalent or dative bond(s) between the ligand and the metal(s). Furthermore, as typically used herein, a by-product which does not “interfere with” or “significantly inhibit” a reaction of interest generally refers to a by-product which reduces the catalytic activity and/or selectivity of the metal-ligand complex, composition or compound being screened, in the reaction of interest, by less than about 80%, preferably less than about 50%, more preferably less than about 20%, and most preferably less than about 10%.

Generally speaking, the advantages of the present process are the result of using a metal precursor that has appropriate ancillary groups. These ancillary groups impart solubility to the metal center, and thereby enable one to prepare solutions of the compound that can be dispensed easily (and preferably through the use of automation) to an array of reaction wells which, for example, may contain various other ligands. The ancillary groups, also referred to as “solublizing groups,” preferably also accommodate coordination of other chemically diverse ligands (e.g., “member” ligands from a ligand library or array). It is to be noted that while it is important that such groups impart solubility to the metal precursor, there is also a balance that must be maintained between solubility, reactivity and stability; that is, generally speaking, groups are employed which act to solubilize the metal precursor, while preferably optimizing both reactivity and stability of the resulting soluble metal precursor. Additionally, it is desirable for the soluble metal precursor to be sufficiently stable, such that it may be stored and handled without decomposing.

Definitions

In addition to other terms and phrases defined elsewhere, the following various terms and phrases as used in the instant specification have the following meanings:

As used herein, the phrase “characterized by the formula” is not intended to be limiting and is used in the same way that “comprising” is commonly used.

The term “independently selected” is used herein to indicate, for example, that an “R” group (e.g., R¹, R², R³, R⁴ and/or R⁵) can be identical or different (e.g., R¹, R², R³, R⁴ and/or R⁵ may all be substituted alkyls, or R¹ and R² may be a substituted alkyl and R³ may be an aryl, etc.). It is to be noted that this term generally applies in the same manner to other substituent designations, as well.

Additionally, a named “R” group will generally have the structure that is recognized in the art as corresponding to R groups having that name.

The terms “compound” and “complex” are generally used interchangeably in this specification, but those of skill in the art may recognize certain compounds as complexes and vice versa. For the purposes of illustration, representative groups are defined herein. However, these definitions are intended to supplement and illustrate, not preclude, the definitions known to those of skill in the art.

The term “alkyl” is used herein to refer to a branched or unbranched, saturated or unsaturated acyclic hydrocarbon radical. Suitable alkyl radicals include, for example, methyl, ethyl, n-propyl, i-propyl, 2-propenyl (or allyl), vinyl, n-butyl, t-butyl, i-butyl (or 2-methylpropyl), etc. In particular embodiments, alkyls have between 1 and 200 carbon atoms, between 1 and 50 carbon atoms or between 1 and 20 carbon atoms.

“Substituted alkyl” refers to an alkyl as just described in which one or more hydrogen atoms bound to any carbon of the alkyl radical or substituent is replaced by another group such as a heteroatom, halogen, aryl, substituted aryl, cycloalkyl, substituted cycloalkyl, and combinations thereof. Suitable substituted alkyls include, for example, benzyl, trifluoromethyl and the like. It is to be noted that the bond between the carbon atom and the heteroatom may be saturated or unsaturated. Thus, an alkyl radical substituted with a heterocycloalkyl, substituted heterocycloalkyl, heteroaryl, substituted heteroaryl, alkoxy, aryloxy, boryl, phosphino, amino, silyl, thio or seleno is within the scope of the term substituted (or “hetero-substituted”) alkyl. Suitable heteroalkyls include cyano, benzoyl, 2-pyridyl, 2-furyl and the like.

The term “heteroalkyl” refers to an alkyl radical or substituent as described above in which one or more carbon atoms of the main chain of the alkyl radical is replaced by a heteroatom selected from the group consisting of N, 0, P, B, S, Si, Sb, Al, Sn, As, Se and Ge.

The term “cycloalkyl” is used herein to refer to a saturated or unsaturated cyclic, non-aromatic hydrocarbon radical having a single ring or multiple condensed rings. Suitable cycloalkyl radicals include, for example, cyclopentyl, cyclohexyl, cyclooctenyl, bicyclooctyl, etc. In particular embodiments, cycloalkyls have between 3 and 200 carbon atoms, between 3 and 50 carbon atoms or between 3 and 20 carbon atoms.

“Substituted cycloalkyl” refers to a cycloalkyl radical or substituent as described above in which one or more hydrogen atoms bound to any carbon of the cycloalkyl radical is replaced by another group such as a heteroatom, halogen, alkyl, substituted alkyl, aryl, substituted aryl, cycloalkyl, substituted cycloalkyl, heterocycloalkyl, substituted heterocycloalkyl, heteroaryl, substituted heteroaryl, alkoxy, aryloxy, boryl, phosphino, amino, silyl, thio, seleno and combinations thereof. Suitable substituted cycloalkyl radicals include, for example: 4-dimethylamino-cyclohexyl; 4,5-dibromocyclohept-4-enyl and the like.

The term “heterocycloalkyl” is used herein to refer to a cycloalkyl radical as described about in which one or more or all carbon atoms of the saturated or unsaturated cyclic radical are replaced by a heteroatom such as nitrogen, phosphorous, oxygen, sulfur, silicon, germanium, selenium, or boron. Suitable heterocycloalkyls include, for example, piperazinyl, morpholinyl, tetrahydropyranyl, tetrahydro-furanyl, piperidinyl, pyrrolidinyl, oxazolinyl and the like.

“Substituted heterocycloalkyl” refers to a heterocycloalkyl radical as described above in which one or more hydrogen atoms bound to any atom of the heterocycloalkyl radical is replaced by another group such as a halogen, alkyl, substituted alkyl, aryl, substituted aryl, heteroaryl, substituted heteroaryl, alkoxy, aryloxy, boryl, phosphino, amino, silyl, thio, seleno and combinations thereof. Suitable substituted heterocycloalkyl radicals include, for example, N-methylpiperazinyl, 3-dimethylaminomorpholinyl and the like.

The term “aryl” is used herein to refer to an aromatic radical or substituent which may be a single aromatic ring or multiple aromatic rings which are fused together, linked covalently, or linked to a common group such as a methylene or ethylene moiety. The aromatic ring(s) may include phenyl, naphthyl and biphenyl, among others. In particular embodiments, aryls have between 1 and 200 carbon atoms, between 1 and 50 carbon atoms or between 1 and 20 carbon atoms.

“Substituted aryl” refers to an aryl radical as described above in which one or more hydrogen atoms bound to any carbon is replaced by one or more functional groups such as alkyl, substituted alkyl, cycloalkyl, substituted cycloalkyl, heterocycloalkyl, substituted heterocycloalkyl, halogen, alkylhalos (e.g., CF₃), hydroxy, amino, phosphino, alkoxy, boryl, silyl, amino, thio, nitro, and both saturated and unsaturated cyclic hydrocarbons which are fused to the aromatic ring(s), linked covalently or linked to a common group such as a methylene or ethylene moiety. The common linking group may also be a carbonyl, as in benzophenone, or oxygen, as in diphenylether, or nitrogen, as in diphenylamine.

The term “heteroaryl” as used herein refers to an aromatic ring substituent or radical in which one or more carbon atoms of the aromatic ring(s) is/are replaced by a heteroatom, such as nitrogen, oxygen, boron, selenium, phosphorus, silicon or sulfur. Heteroaryl refers to structures that may be a single aromatic ring, multiple aromatic ring(s), or one or more aromatic rings coupled to one or more non-aromatic ring(s). In structures having multiple rings, the rings can be fused together, linked covalently, or linked to a common group, such as a methylene or ethylene moiety. The common linking group may also be a carbonyl, as in phenyl pyridyl ketone. As used herein, rings such as thiophene, pyridine, isoxazole, phthalimide, pyrazole, indole, furan, etc., or benzo-fused analogues of these rings, are defined by the term “heteroaryl.”

“Substituted heteroaryl” refers to a heteroaryl radical as described above in which one or more hydrogen atoms bound to any atom of the heteroaryl moiety is replaced by another group such as a halogen, alkyl, substituted alkyl, aryl, substituted aryl, heteroaryl, substituted heteroaryl, alkoxy, aryloxy, boryl, phosphino, amino, silyl, thio, seleno and combinations thereof. Suitable substituted heteroaryl radicals include, for example, 4-N,N-dimethylaminopyridine.

The term “alkoxy” refers to a “−OZ¹” radical, where Z¹ is selected from the group consisting of alkyl, substituted alkyl, cycloalkyl, substituted cycloalkyl, heterocylcoalkyl, substituted heterocycloalkyl, silyl, hydrogen and/or combinations thereof as described herein. Suitable alkoxy radicals include, for example, methoxy, ethoxy, benzyloxy, t-butoxy, etc. A related term is “aryloxy” where Z¹ is selected from the group consisting of aryl, substituted aryl, heteroaryl, substituted heteroaryl, and combinations thereof. Examples of suitable aryloxy radicals include phenoxy, substituted phenoxy, 2-pyridinoxy, 8-quinalinoxy and the like.

As used herein the term “silyl” refers to a “−SiZ¹Z²Z³” radical, where each of Z¹, Z² and Z³ is independently selected from the group consisting of alkyl, substituted alkyl, cycloalkyl, heterocycloalkyl, heterocyclic, aryl, substituted aryl, heteroaryl, substituted heteroaryl, alkoxy, aryloxy, amino, silyl, hydrogen and combinations thereof.

As used herein the term “boryl” refers to a “−BZ¹Z²” radical or group, where each of Z¹ and Z² is independently selected from the group consisting of alkyl, substituted alkyl, cycloalkyl, heterocycloalkyl, heterocyclic, aryl, substituted aryl, heteroaryl, substituted heteroaryl, alkoxy, aryloxy, amino, silyl, hydrogen and combinations thereof.

As used herein, the term “phosphino” refers to a “−PZ¹Z²” radical or group, where each of Z¹ and Z² is independently selected from the group consisting of hydrogen, substituted or unsubstituted alkyl, cycloalkyl, heterocycloalkyl, heterocyclic, aryl, substituted aryl, heteroaryl, silyl, alkoxy, aryloxy, amino and combinations thereof.

The term “amino” refers to a “−NZ¹Z²” radical or group, where each of Z¹ and Z² is independently selected from the group consisting of hydrogen, alkyl, substituted alkyl, cycloalkyl, substituted cycloalkyl, heterocycloalkyl, substituted heterocycloalkyl, aryl, substituted aryl, heteroaryl, substituted heteroaryl, alkoxy, aryloxy, silyl, boryl and combinations thereof.

The term “thio” refers to a “−SZ¹” radical or group, where Z¹ is selected from the group consisting of hydrogen, alkyl, substituted alkyl, cycloalkyl, substituted cycloalkyl, heterocycloalkyl, substituted heterocycloalkyl, aryl, substituted aryl, heteroaryl, substituted heteroaryl, alkoxy, aryloxy, silyl and combinations thereof.

The term “seleno” refers to a “−SeZ¹” group or radical, where Z¹ is selected from the group consisting of hydrogen, alkyl, substituted alkyl, cycloalkyl, substituted cycloalkyl, heterocycloalkyl, substituted heterocycloalkyl, aryl, substituted aryl, heteroaryl, substituted heteroaryl, alkoxy, aryloxy, silyl and combinations thereof.

The term “saturated” refers to the lack of double and triple bonds between atoms of a radical group, such as for example ethyl, cyclohexyl, pyrrolidinyl and the like.

The term “unsaturated” refers to the presence of one or more double and/or triple bonds between atoms of a radical group, such as for example vinyl, acetylide, oxazolinyl, cyclohexenyl, acetyl and the like.

Abbreviations used herein include the following: Cp=η⁵-cyclopentadienyl; Cp*=η⁵-pentamethylcyclopentadienyl; MAO=methylaluminoaoxane; DME=dimethoxyethane; DEAD=diethylazodicarboxylate; COD=cyclooctadiene; DBU=1,8-diazabycicylo[5,4,0]undec-7-ene; BF15=B(C₆F₅)₃; anilBF20 or aniliniumBF20=[PhNMe₂H]⁺[B(C₆F₅)₄]⁻; tritylBF20=[Ph₃C] [B (C₆F₅)₄]; TEAL=triethylaluminum; TIBA=tri(isobutyl)aluminum; TFA=CF₃CO₂; OTs=p-MeC₆H₄SO₃; TMA=trimethylaluminum; TMEDA=N,N,N′,N′-tetramethylethylene diamine; cy=cyclohexyl; mes or mesityl=2,4,6-trimethylphenyl; acac=2,4-pentanedionate; dba=dibenzylidene acetone; OTf=triflate (“OSO₂CF₃”) ; OAc=acetate; COT=cyclooctatetraene; hfac=hexafluoroacetylacetanoate; and, [BAr_{f4}]=tetrakis(3,5-bis (trifluoromethy)phenyl)borate.

Soluble Metal Precursors

In accordance with the present process, an array of metal-ligand compositions is prepared by combining one or more metals, in the form of dissolved, soluble metal precursors, with a plurality of member ligands (e.g., unbound ligands, or ligands free of covalent or dative bonds to a solid support) from a ligand array. Unlike combinatorial processes heretofore utilized to prepare an array or library of metal-ligand compositions in which a metal, in solid or slurry form, is combined with ligands, the process of the present invention utilizes solublizing groups to impart solubility to the metal, in order that it may be handled in solution form. Such a process is advantageous not only because it is more conducive to automation, but also because it enables very small quantities of the solublized metal to be precisely delivered to a site where it can be combined with a desired amount of a member ligand from a ligand array. For example, in some instances it may be desirable to combine one or more stoichiometric equivalents of a soluble metal precursor with a member ligand (e.g., a molar ratio of about 0.5:1 to about 4:1 of the metal precursor to member ligand, from about 1:1 to about 3:1, or from about 1.5:1 to about 2:1).

In a first embodiment, the soluble metal precursors of the present invention may be generally represented by the empirical formula:

MRn

wherein: M represents a metal atom, such as a metal atom selected from the group consisting of transition metals, lanthanide metals, actinide metals or main group metals; R represents one or more solublizing groups attached to the metal atom; and, n represents the number of R groups (which may be the same or different) attached to or associated with the metal. Typically, each R group is independently selected from the group consisting of halides (e.g., Cl, F, I or Br), alkyl, substituted alkyl, cycloalkyl, substituted cycloalkyl, heterocycloalkyl, substituted heterocycloalkyl, allyl, substituted allyl, alkylidene, alkylidyne, cyclopentadienyl, substituted cyclopentadienyl, aryl, substituted aryl, heteroaryl, substituted heteroaryl, alkoxy, aryloxy, hydroxy, boryl, silyl, hydride, thio, seleno, phosphino, amino, carboxylates, 1,3-dionates, oxalates, carbonates, nitrates, sulfates, perchlorates, sulfonates, phosphonates, oxos, imidos, sulfidos and combinations thereof. It is to be noted that, in some embodiments, depending upon the nature of the metal and the R groups present, the soluble metal precursor may be in the form of a dimer, trimer, or higher order structure, wherein there may be two or more metal atoms that are bridged by one or more R groups. Additionally, in some embodiments there may or may not be metal-metal bonds present.

The number of R groups per metal center (i.e., the value of n) will depend for example on the identity of the metal, the oxidation state of the metal, the identity or nature of each of the R groups, and the overall charge on the soluble metal precursor complex. Theoretically, however, there may be up to about 7 or 8 R groups per metal atom in the metal precursor (e.g., about 1, 2, 3, 4, 5, 6, 7 or 8 R groups may be bonded to the metal atom). Additionally, two or more R groups may be connected together with a linking group to form chelating groups.

In a second embodiment, the soluble metal precursor of the present process may be generally represented by the formula:

MRₙLₘ

wherein: M and R are as defined above; L represents one or more neutral solublizing groups attached to the metal atom; n ranges from about 0 to about 7; and, m ranges from about 1 to about 6, provided the sum of n and m is at least 1. Typically, each L group is independently selected from the group consisting of carbon monoxide, isocyanide, nitrous oxide, alkylnitriles (i.e., compounds having the general formula “NCR,” such as acetonitrile, where R=methyl), arylnitriles, η⁶-arenes (such as η⁶-p-cymene), olefins (including cyclic olefins, such as cyclooctene, and bicyclic olefins, such as norbornene), dienes (including cyclic dienes, such as 1,5-cyclooctadiene, and bicyclic dienes, such as norbornadiene), trienes (such as cycloheptatriene), cyclooctatetraenes, alkynes, as well as PX₃, NX₃, OX₂, SX₂, SeX₂, OPX₃, ONX₃, SPX₃, OSX₂, OSOX₂, X₂N-NX₂, XS-SX, XO-OX and combinations thereof, wherein each X is independently selected from a group consisting of alkyl, substituted alkyl, heteroalkyl, substituted heteroalkyl, cycloalkyl, substituted cycloalkyl, heterocycloalkyl, substituted heterocycloalkyl, aryl, substituted aryl, heteroaryl, substituted heteroaryl and silyl. optionally, two or more X groups may be linked to form one or more ring structures with the other atoms; thus, for example, OX₂ includes tetrahydrofuran. Bicyclic rings are included, as well.

It is to be noted that the number of L groups bound to the metal is dependent on the identity of the metal, the oxidation state of the metal and the identity of each of the groups chosen for R and L. In theory, there may be up to about 6 L groups attached to the metal atom in the metal precursor, meaning that about 1, 2, 3, 4, 5 or 6 L groups may be datively bonded to the metal atom. Typically, however, m ranges from about 1 to about 5, with n+m being less than about 7 when M is a transition metal and n+m being less than about 9 when M is a lanthanide metal.

It is to be further noted that two or more L groups may be connected together with a linking group to form chelating neutral groups, such as, for example, in the case of TMEDA (N,N,N,N′-tetramethylethylenediamine).

In a third embodiment, the metal precursor may be characterized by the empirical formula:

MₚR_{n′}L_{m′}

wherein: M, R and L are as defined above; n′ and m′ are each independently numbers greater than or equal to about 0, with the proviso that the sum of n′ and m′ is great than or equal to about 2; and, p is a number greater than 1, each M being independently selected from the list of metals provided herein (that is, when multiple metal atoms are present they may be the same or different).

It is to be noted that, in some embodiments, one or more of the R groups of the soluble metal precursor are preferably large or bulky groups. Without being held to any particular theory, some bulky R groups are generally preferred because they are believed to impart additional stabilization to the metal precursor against degradation, and also act to increase solubility of the metal precursor. Bulky R groups include groups such as trimethylsilyl-substituted alkyl groups (such as mono-, bis- and tris-(trimethylsilyl) methyl), substituted aryl groups (such as mesityl), alkoxy (such as tert-butoxy), aryloxy (such as 2,6-bis(tert-butyl)phenoxy), bulky amino (such as N,N-bis(trimethylsilyl) amino), 1,3-dionates (including 2,4-pentanedionate, as well as the substituted versions thereof, such as 2,2,6,6-tetramethyl-3,5-heptanedionate), and carboxylates (such as trifluoroacetate).

It is to be further noted that, in some embodiments, the metal precursor is a homoleptic compound, meaning that each R group is the same. Such embodiments include those wherein each R group is the same and selected from the group consisting of, for example, halide, hydride, alkyl, substituted alkyl, cycloalkyl, substituted cycloalkyl, aryl, substituted aryl, heteroalkyl, substituted heteroalkyl, alkoxy, aryloxy, 1,3-dionates, carboxylates, sulfonates and amino.

It is to be still further noted that, in some embodiments, substitutents may be present on one or more of the solublizing R groups, which may act to impart additional stability to the metal precursor by binding to the metal center, such as via an amino or ether substituent. Examples of such embodiments include 2-dialkylaminobenzyl groups or 2-dialkylaminomethylphenyl groups, such as in the soluble metal precursors “M(η²—C₆H₄CH₂NMe₂)₂,” wherein M is typically manganese, iron, cobalt, nickel or palladium, among others, and η12 represents bidentate coordination.

In some cases, the soluble metal precursor may be ionic in nature; that is, the metal-centered complex (i.e., MRₙ or MRₙLₘ or MₚR_{n′}L_{m′}) may be charged. In such cases, the solubilized metal precursors may additionally comprise one or more counterions having an appropriate counter balancing charge. Examples of such ionic species include: (i) anionic, solubilized metal precursor species, such as Co(mesityl)₃⁻, which may be appropriately counter balanced for example with a cation such as Li(THF)₄⁺; and, (ii) cationic, solubilized metal precursor species in which the positively charged metal-centered complex is counter balanced with an anionic species, such as for example in the case of [Mn(mesityl) (OEt₂)₃⁺] [B(C₆H₅)₄⁻] or more preferably [(COD) PdMe (CH₃CN) ]⁺[BAr_{f4}]⁻.

In some preferred embodiments of the soluble metal precursors useful in this invention, the R and L groups are chosen such that the reaction of the soluble metal precursor with other reagents employed in the reaction, especially with member ligands, produces: (i) no by-product; (ii) only chemically innocuous by-products (i.e., by-products that do not interfere with or significantly inhibit the desired reaction of interest); or, (iii) potentially detrimental by-products that can be easily removed or easily made to be innocuous. For example, Lewis base by-products (i.e., by-products wherein a Lewis base solublizing group, or a derivative thereof, is displaced from the soluble metal precursor) may bind to the catalytic metal center and impair the performance of the catalyst in the chemical reaction of interest. Such Lewis base by-products (e.g., pyridine or phosphines) may be rendered innocuous by the addition of a Lewis acid reagent (e.g., B(C₆F₅)₃) that is capable of reacting with the given Lewis base to form a Lewis acid-Lewis base reaction product that does not interfere with or significantly inhibit the screening reaction of interest. Alternatively, by-products may be removed prior to the reaction of interest; for example, volatile by-products may alternatively be removed by evaporation (under reduced pressure, for example).

In view of the foregoing, preferred solublized metal precursors include the following:

| | |
|---|---|
| M(CH₂Ph)₄, | [M(mesityl)₃(THF), |
| M(CH₂SiMe₃)₃, | M(CH₃)₃(Cl)₂, |
| M(NMe₂)₃(Cl)₂, | M(CH₂SiMe₃)₄, |
| M(mesityl)₂(THF), | M(mesityl)₂(THF)₃, |
| [M(mesityl)₂]₂, | M(mesityl)₃M(THF)₄, |
| [M(mesityl)₂]₃, | M(mesityl)₃, |
| M(CH(SiMe₃)₂)₃, | M(O(2,6-(tBu)₂C₆H₃))₃, |
| M(O(2,6-(tBu)₂4-Me—C₆H₃))₃, | M(N(SiMe₃)₂)₃, |
| M(acac)₂, | M(acac)₃, |
| M(O)(acac)₃, | [(COD)MMe(CH₃CN)]⁺[BArₜ₄], |
| M(TFA)₂, | M(TFA)₃, |
| [M(TFA)₂]₂, | MCl₃(THF)₃, |
| (COD)MMeCl, | [(cyclooctene)MMeCl]₂, |
| (COD)MMeOTf, | [(allyl)MCl]₂, |
| [(CH₃O₂CC₃H₄)MBr]₂, | [(allyl)M(TFA)]₂, |
| (p-cymene)M(TFA)₂(CH₃CN), | (p-cymene)M(mesityl)(TFA), |
| (PPh₃)₄MH₂, | (PPh₃)₂M(Ph)Cl, |
| (PPh₃)₄M, | (COD)₂M, |
| (py)₂M(CH₂SiMe₃)₂, | M(C(SiMe₃)₃)₂, |
| M(CH₂CMe₃)₄, | M(NMe₂)₄, |
| M(NEt₂)₄, | M(NMe₂)₂Cl₂, |
| M(N(SiMe₃)₂)₂Cl₂, | M(TFA)₄, |
| M(TFA)₂Cl₂, | MCl₃(THF)₃, |
| M(CH(SiMe₃)₂)₃(THF), | M(O-2,6-iPr—C₆H₃)₄M″(THF), |
| M(NMe₂)₅, | (TMEDA)MMe₂, |
| M(CH₂CMe₃)₂Cl₃, | MPh₅, |
| M(Ph₃P)₃CH₃, | [M(Ph₃P)₃H]₂N₂, |
| M(dba)₂, | M₂(dba)₃, |
| M(OAc)₂, | [M(TFA)₂]₂, |
| M(NMe₂)₂Cl₂, | M(octanoate)₂, |
| [[CH₂CHSi(CH₃)₂O]₃M2, | [M(PCy₃)₂]₂N₂, and |
| M(O(2,6-(tBu)₂-4-tBu—C₆H₃))₃ | |

(where M″ is an alkali metal, typically Li, Na, K, Cs or Rb and M is a metal selected from the groups noted herein).

In this regard, it is to be noted that in some embodiments, metal precursors useful in this invention may be prepared and isolated in a form in which solvent residue is entrained in the isolated product, the solvent residue may or may not be bound to the metal center. The metal precursor Ni(TFA)₂(OEt₂)_{0.48} is an example of such an embodiment. For the purposes of this invention, such entrained solvent residue will not be included in the formulae used to describe the metal precursors.

Among the soluble metal precursors noted above, the metal-trifluoroacetate compounds (i.e., “M(TFA)ₙ,” where TFA =⁻O₂CCF₃), are preferred in some embodiments, particularly in the screening of polyolefin polymerization reactions. Particularly preferred compounds including Ni(TFA)₂, Fe(TFA)₂, Fe(TFA)₃, Co(TFA)₂, Mn(TFA)₂, [Cr(TFA)₂]₂, Cr(TFA)₃ and V(TFA)₃. The trifluoroacetate group, ⁻O₂CCF₃, has a number of advantages over other anionically charged groups such as halides. Traditionally, the metal halide precursors have been the most common metal precursors used to prepare transition metal based catalysts, especially for olefin polymerization applications. However, unlike many transition metal halide compounds, transition metal trifluoroacetate complexes are soluble in most organic solvents, especially in the presence of a small amount of an ether solvent, such a THF or diethyl ether. This enables solutions of transition metal trifluoroacetate complexes to be easily prepared and handled in parallel or rapid serial synthesis and screening applications.

The much higher solubility of many transition metal trifluoroacetate complexes compared with halide analogs may also significantly enhance the rates of reaction with ligands and ligand arrays. Like halide groups, trifluoroacetate groups are monoanionic, and are capable of binding to a metal center in a monodentate fashion. Also, like halide groups, trifluoroacetate groups are capable of bridging between two metal centers or acting as non-coordinating counterions. However, trifluoroacetate groups can also bind to the metal center in a bidentate fashion, via both oxygen atoms. Trifluoroacetate groups can easily switch between different metal-binding modes, thereby enabling them to occupy or vacate coordination sites on the metal center to accommodate the coordination demands of a given ligand. The trifluoroacetate group is also capable of undergoing metathesis reactions with sources of alkyl, aryl and hydride groups such as Grignard reagents, alkyl- and aryl-lithium compounds, and alkyl- and aryl-aluminum compounds (including alumoxanes). This enables them to be useful in the preparation of catalysts for reactions, such as olefin polymerizations and copolymerizations.

There are other charged groups, such as tosylate (p-CH₃C₆H₄SO3⁻) and triflate (CF₃SO₃⁻), that also impart solubility to metal precursor compounds and may have variable modes of coordination. Compounds such as M(p-CH₃C₆H₄SO₃)ₙ and M(CF₃SO₃)ₙ can be prepared for many metals in a variety of oxidation states, analogous to their metal-trifluoroacetate counterparts. Neutral groups such as alkylnitriles (“RCN”, such as wherein R=methyl), pyridines and phosphines are commonly used in the preparation of metal precursors, such as ((CH₃)₃CN)₂PdCl₂, (pyridine)₄NiCl₂ and (PPh₃)₂NiCl2, because of their ability to solubilize and stabilize the metal center in a particular oxidation state, geometry or coordination environment. Metal precursors containing these ancillary neutral groups vary in their ability to undergo substitution reactions with ligands. Such displaced neutral groups may need to be treated prior to the screening step to render them innocuous, such that to they do not interfere with or significantly inhibit the subsequent screening reaction of interest (such as catalytic transformation reactions).

Some examples of homoleptic metal-alkyl compounds that are useful as soluble metal precursors in this invention include the complex M(C(SiMe₃)₃)₂, where M=Co, Fe, Mn, Cr. The bulky alkyl groups C(SiMe₃)₃ provide for solubility in solvents such as hydrocarbons, and also kinetically stabilize metal centers with very low coordination numbers. Metal precursors of this type can react with neutral ligands via an associative process. They can also react with charged ligands that contain acidic protons through the elimination of CH(SiMe₃)₃, an innocuous, volatile and easily removed by-product.

Examples of a useful soluble metal precursor for this invention, characterized by the formula MRₙLₘ, include the soluble Lewis base-stabilized, metal-alkyl precursor (pyridine)₂Ni(CH₂SiMe₃)₂, which contains neutral pyridine groups and bulky alkyl groups, CH₂SiMe₃, that provide for solubility in solvents such as hydrocarbons and also kinetically stabilize the metal center. This metal precursor can react with neutral ligands (e.g. (2,0) or (3,0) ligands, wherein as denoted below the first number represents the coordination number and the second number represents the charge of the ligand) via displacement of one or more of the pyridine groups, or with protonated versions of charged ligands (e.g. (2,−1) or( 3,−1) ligands, wherein as denoted below the first number represents the coordination number and the second number represents the charge of the ligand) via elimination of Si(CH₃)₄ and one or more pyridine groups.

While the metal atom M of the soluble metal precursor may be essentially any transition metal, lanthanide metal, actinide metal or main group metal atom, preferred metals include Sc, Ti, V, Cr, Mn, Fe, Co, Ni, Cu, Zn, Y, Zr, Nb, Mo, Ru, Rh, Pd, Ag, Cd, La, Hf, Ta, W, Re, Os, Ir, Pt, Au, Hg, Ce, Pr, Nd, Sm, Eu, Gd, Tb, Dy, Ho, Er, Tm, Yb, Lu, U, Th, Al, Ga, Tl, In, Li, Na, K, Rb, Cs, Be, Mg, Ca, Sr, Ba, Ge, Sn, Pb, Sb and Bi. More preferably, however, the metal atom is selected from the group consisting of Ti, Zr, Hf, V, Ta, Nb, Cr, Fe, Co, Sc, Y, La, Ce, Pr, Nd, Sm, Eu, Gd, Tb, Dy, Ho, Er, Tm, Yb, Lu, Ni, Pd, Mn, Ru, Rh, Pt and Cu.

In view of the foregoing, it is to be noted that preferred soluble metal precursors include, among others:

| | |
|---|---|
| Ti(CH₂Ph)₄, | Zr(CH₂Ph)₄, |
| Hf(CH₂Ph)₄, | V(mes)₃(THF), |
| Ta(CH₃)(Cl)₂, | Nb(CH₃)₃(Cl)₂, |
| Ta(NMe₂)₃(Cl)₂, | Cr(CH(SiMe₃) ₂)₃, |
| Cr(mesityl)₂(THF), | Cr(mesityl)₂(THF)₃, |
| [Fe(mesityl)₂]₂, | [Co(mesityl)₂]_{2,} |
| Co(mesityl)₃Li(THF)₄, | [Mn(mesityl)₂]₃, |
| Cr(mesityl)₃, | Sc(CH(SiMe₃)₂)₃, |
| Y(CH(SiMe₃)₂)₃, | Ln(CH(SiMe₃)₂)₃, |
| Sc(O(2,6-(tBu)₂C₆H₃))₃, | Y(O(2,6-(tBu)₂C₆H₃))₃, |
| Ln(O(2,6-(tBu)₂C₆H₃))₃, | Sc(O(2,6-(tBu)₂4-Me-C₆H₃))₃, |
| Y(O(2,6-(tBu)₂4-Me—C₆H₃))₃, | Ln(O(2,6-(tBu)₂4-Me-C₆H₃))₃, |
| Sc(N(SiMe₃)₂)₃, | Y(N(SiMe₃)₂)₃, |
| Ln(N(SiMe₃)₂)₃, | Ni(acac)₂, |
| Pd(acac)₂, | Co(acac)₃, |
| Fe(acac)₃, | Fe(acac)₂, |
| Mn(acac)₂, | Cr(acac)₂, |
| Cr(acac)₃, | V(acac)₃, |
| V(O)(acac)₃, | Ni(TFA)₂, |
| Fe(TFA)₂, | Fe(TFA)₃, |
| Co(TFA)₂, | Mn(TEA)₂, |
| [Cr(TEA)₂]₂, | Cr(TFA)₃, |
| V(TFA)₃, | (COD)PdMeCl, |
| [(cyclooctene)PdMeCl]₂, | (COD)PdMeOTf, |
| [(allyl)PdCl]₂, | [(allyl)NiCl]₂, |
| [(CH₃O₂CC₃H₄)NiBr]₂, | [(allyl)Ni(TFA)]₂, |
| (p-cymene)Ru(TFA)₂(CH₃CN), | (p-cymene)Ru(mesityl)(TFA), |
| (PPh₃)₄RuH₂, | (PPh₃)₂Ni(Ph)Cl, |
| (PPh₃)₄Ni, | (COD)₂Ni, |
| (py)₂Ni(CH₂SiMe₃)₂, | Fe(C(SiMe₃)₃)₂, |
| Co(C(SiMe₃)₃)₂, | Mn(C(SiMe₃)₃)₂, |
| Ti(CH₂CMe₃)₄, | Zr(CH₂CMe₃)₄, |
| Hf(CH₂CMe₃)₄, | Ti(NMe₂)₄, |
| Co(PMe₃)₄Me, | Zr(NMe₂)₄, |
| Hf(NMe₂)₄, | Zr(NEt₂)₄, |
| Ti(NMe₂)₂Cl₂, | Zr(N(SiMe₃)₂)₂Cl₂, |
| Hf(N(SiMe₃)₂)₂Cl₂, | Zr(TFA)₄, |
| Hf(TFA)₄, | Ti(TFA)₂Cl₂, |
| TiCl₃(THF)₃, | V(CH(SiMe₃)₂)₃(THF), |
| V(O-2,6-iPr-C₆H₃)₄Li(THF), | Ta(NMe₂)₅, |
| (TMEDA)NiMe₂, | (TMEDA)PdMe₂, |
| Ta(CH₂CMe₃)₂Cl₃, | TaPh₅, |
| Co(Ph₃P)₃CH₃, | [Co(Ph₃P)₃H]₂N₂, |
| [Ni(PCy₃)₂]₂N₂, | ((CH₃)₃CN)₂PdCl₂ |
| Pd(dba)₂, | Pd₂(dba)₃, |
| Pd(OAc)₂, | [Cr(TFA)₂]₂, |
| Ti(NMe₂)₂Cl₂, | Ni(octanoate)₂, |
| [(COD)PdMe(CH₃CN)]⁺BAr_{f4}⁻, | [[CH₂CHSi(CH₃)₂]₂O]₃M*₂ |

where

$$Ln = \text{La, Ce, Pr, Nd, Sm, Eu, Gd, Tb, Dy, Ho, Er, Tm, Yb or Lu}$$

$$and$$

$${M}^{*} = \text{Pd or Pt.}$$

Ligands

Ligands which are suitable for use in the present invention, for purposes of combining with a soluble metal precursor to form metal-ligand compositions or compounds, are generally those ligands that bind metal atoms or ions (e.g., via covalent bonds, dative bonds or combinations thereof). A plethora of such metal-binding ligands are known in the art. (See, for example, Collman, J. P., et al. PRINCIPLES AND APPLICATIONS OF ORGANOTRANSITION METAL CHEMISTRY, University Science Books, California, 1987, and references cited therein which are herein incorporated by reference.) Ligand characteristics that can be varied include, but are not limited to, the number of coordination sites on the metal which the ligand can occupy, the charge and electronic influence of the ligand, the geometry imposed on the metal by the ligand, the size and/or shape of the ligand, etc. The metal-ligand compositions which form may have more than one geometry.

Although essentially any ligand may be employed in the present process, generally speaking the ligands will have about 1, 2, 3 or 4 sites capable of coordinating to a metal center, while the charge will be about 0, −1, −2 or −3. In this regard it is to be noted that, in one embodiment, the “charge” on the ligand refers to the number of non-dative covalent bonds that could be formed with the metal center. In another embodiment, “charge” refers to the charge that one skilled in the art would assign to the ligand to balance the overall charge of the metal-ligand complex when the metal center is considered to be an ion with a positive charge that is equivalent to the oxidation state of the metal, and may be represented by M^{Z+}, with M being the metal and z being the oxidation state. It is to be further noted that in some embodiments it is preferred that the ligand coordinate with the metal to form a 5 membered ring or larger. Without being held to a particular theory, it is generally believed that, in at least some embodiments, at least about a 5 membered ring is preferred because smaller rings (e.g., 4 membered or even 3 membered) are more geometrically limited, in part due to higher bond strain, and as a result are less thermodynamically favorable.

It is to be noted that, due to the nature of their structure, certain ligands will have more than one possible coordination number and/or more than one possible charge. Also, a ligand may be deprotonated prior to use with the metal precursor or it may be deprotonated upon reaction with the metal precursor, for example upon reaction with MRₙ to eliminate RH in the process of forming the metal-ligand complex or compound. Examples of ligands that can be used in the present invention include, but are not limited to, the following:

(1) One-site, monoanionic ligands which include, for example: mono-Cp ligands, such as those that might form a complex like Cp*MR₂⁺A⁻ (wherein Cp* and R are as defined herein, and A⁻=anion of A, A being as defined here and generally being any negatively charged group;

for example, A may be selected from those groups listed for R, when not datively or covalently bound to a metal M, or the list of boron-containing compounds provided herein below) ; and, other mono-Cp systems, such as aryloxy, that might form a complex like (aryloxy)MR₂⁺A⁻;

(2) Two-site, dianionic ligands which include, for example: mono-Cp systems, where a heteroatom based ancillary ligand occupies the second site (see, e.g., U.S. Pat. No. 5,064,802, the teachings of which are incorporated herein by reference); and, non-Cp amide systems (see, e.g., U.S. Pat. Nos. 5,318,935, 5,495,036 and J. Am. Chem. Soc. 1996, 118:10008-10009, the teachings of which are incorporated herein by reference);

(3) Two-site, monoanionic ligands including, for example: those that might form a complex like (CpL)MR⁺A⁻ (wherein L is as defined herein, but here is covalently linked to the cyclopentadienyl group, which may also include other substituents) and related systems (see, e.g., PCT Application No. WO 96/13529, the teachings of which are incorporated herein by reference); or, mono-Cp systems, where a heteroatom based ancillary ligand occupies the second site (see, e.g., European Patent Application No. 0 805 142 A1, as well as PCT Application Nos.

WO 97/42232 and WO 97/42239, each of which are incorporated herein by reference).

(4) Two-site, neutral ligands;

(5) Three-site, neutral ligands;

(6) Three-site, monoanionic ligands;

(7) Three-site, dianionic ligands (an example of which is referred to in Organometallics 1995, 14:3154-3156, which is incorporated herein by reference);

(8) Four-site, neutral, monoanionic and dianionic ligands; and,

(9) Ligands wherein the charge is greater than the number of sites it occupies (see, for example, U.S. Pat. No. 5,504,049, the teachings of which are incorporated herein by reference).

More examples of the types of ligands described above may be found by those of skill in the art in, for example, Gibson, et al., Angew. Chem. Int. Ed., 1999, vol. 38, pp. 428-447, which is incorporated herein by reference.

In some embodiments, the coordination site number of the ligand is greater than the charge. Preferred combinations include those wherein the coordination numbers (CN) of the ligand are independently selected from 1, 2, 3 or 4, and the charge on the ligands are independently selected from 0, −1 or −2. Preferred coordination numbers and charges include:

(i) CN=2, charge=−2;

(ii) CN=2, charge=−1;

(iii) CN=1, charge=−1;

(iv) CN=2, charge=0;

(v) CN=3, charge=−1;

(vi) CN=3, charge=−2;

(vii) CN=3, charge=0;

(viii) CN=4, charge=0;

(ix) CN=4, charge=−1;

(x) CN 4, charge=−2; and,

(xi) CN=1, charge=0.

In other embodiments, the ligand has a charge which is greater than the coordination number, such as for example where CN=1 and charge=−2 (see, e.g., imido ligands referred to in Gibson et al., Id.) . Other examples of such ligands include alkylidene (e.g., R₂C²⁻, in which C forms a formal double bond to a metal center), imido (e.g., RN2−), oxo (O²⁻), or alkylidyne (e.g., RC³⁻, in which C forms a formal triple bond to a metal center).

In this regard it is to be noted that the format used herein to describe classes of ligands lists the coordination number first and the ligand charge second. For example, the notation (2,−2) refers to a ligand having a coordination number (CN) of 2 and a charge of −2. It is to be further noted that, in instances wherein multiple ligands are associated with the metal center, the notation

{(a,0)ᵢ(b,c)_{j}}

is used. In this notation, or formula, (a,0) is directed toward neutral ligands (wherein CN=a and charge=0), which may be provided by an atom with a lone pair of electrons such as, for example, O, N, P, S or C with appropriate other substituents (e.g., carbenes when the atom is C), or by a bond (such as an in an agostic interaction or a pi (π) bond). The second part of this formula (b,c) is directed toward charged ligands (CN=b and charge=c), which may be provided by one or more atoms such as, for example, C, S, O, N, P, B, Si, Se, As, Te, with appropriate other substituents, or by a bond (such a pi (π) bond). The number of neutral ligands bound to the metal center is given by subscript i, and the number of charged ligands bound to the metal center is given by subscript j. Appropriate values of a, b, c, i and j will vary, depending on the metal identity, the oxidation state of the metal, the ligand identities, and whether the metal-ligand complex is neutral or ionic, and will be governed by those chemical principles known to those of skill in the art, including principles such as steric interactions as well as electronic configurations, valency, and oxidation states.

Selected examples of ligands that may be used in this invention, and showing the appropriate notation (CN, charge), are shown below. It is to be noted that the “charged” ligands are shown here in their protonated form, but they may be de-protonated as further described herein.

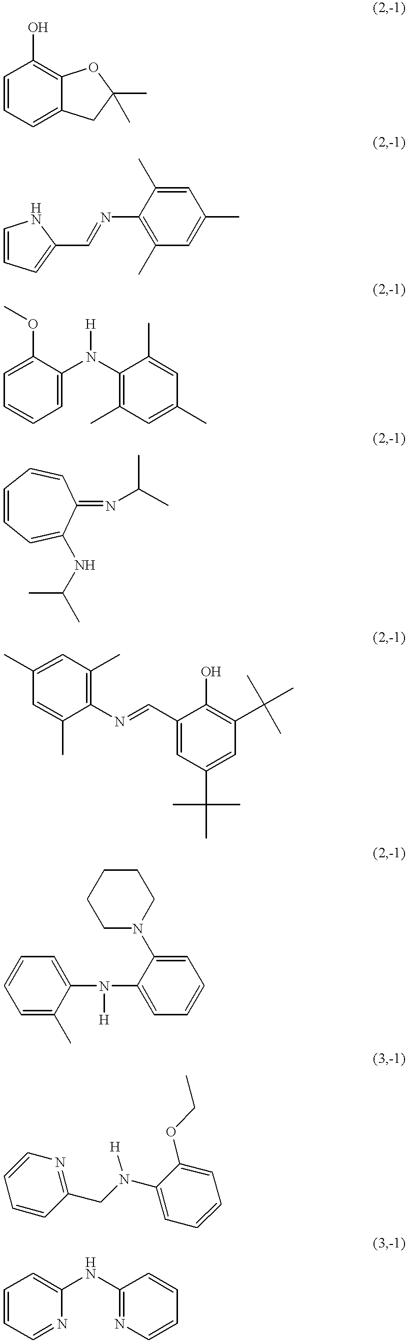

It is to be noted that, as further described herein, when a ligand is mixed with a soluble metal precursor, a metal-ligand composition results, which may be (i) a metal-ligand compound or complex (wherein a covalent or dative bond, or a combination thereof, is formed between the ligand and the metal), or (ii) a mixture comprising a ligand and the soluble metal precursor (wherein no covalent or dative bond is formed). In those instances where no covalent or dative bond is formed, it is to be further noted that such bonds may form upon the addition of some other additive (such as one or more activators, additives and/or modifiers, reactants in the screening reaction of interest, or a combination thereof). In those instances where a metal-ligand compound or complex is formed, it is to be further noted that, depending on the ligand or ligands chosen, the metal complex may take the form of dimers, trimers, or higher orders thereof, or there may be two or more metal atoms that are bridged by one or more ligands. The exact nature of the metal complex(es) or compound(s) formed depends on the exact chemistry of the ligand and the method of combining the soluble metal precursor and ligand, such that a distribution of metal complexes or compounds may form with the number of ligands bound to the metal being greater or less than the number of equivalents of ligands added relative to an equivalent of metal precursor.

Additionally, it is to be noted that, in some embodiments, the ligands may be supported, with or without the metal coordinated, on an organic or inorganic support, (as referred to in, for example: U.S. Pat. No. 6,030,917 and U.S. application Ser. No. 09/025,841, filed Feb. 19, 1998, which are incorporated herein by reference). Suitable supports include silicas, aluminas, clays, zeolites, magnesium chloride, polyethyleneglycols, polystyrenes, polyesters, polyamides, peptides and the like. optionally, the polymeric supports may be cross-linked.

Metal Complexes

In accordance with the process of the present invention, a dissolved, soluble metal precursor is combined with one or more ligands, resulting in the formation of a metal-ligand composition (i.e., a soluble metal precursor-ligand mixture, or a metal-ligand complex or compound). In one embodiment, such complexes or compounds may be characterized by the general empirical formula:

{(a,0)ᵢ(b,c)ⱼ}MR_{n+(jc)}

wherein: a, b, c, i, j, M and R each have the above definitions; and, jc is the product of j multiplied by c. The coordination of the ligand or ligands to the metal atom will be governed by those chemical principles known to those of skill in the art, including principles such as steric interactions as well as electronic configurations. If the above complex bears a charge of w, then the above formula would be expressed as:

[{(a,0)ᵢ(b,c)ⱼ}MR_{n+(jc)-w}]^{W}

wherein w is typically about −3, −2, −1, 1, 2, or 3. When activated with an ion-forming activator (as further described herein), the metal complexes may be characterized by the formula:

[{(a,0)ᵢ(b,c)ⱼ}MR_{n-1+(jc)}]⁺[A]⁻

wherein each of the variables in the above formula are as defined herein (for example, as further discussed herein, A may be B(C₆F₅) ₄) . Moreover, since some embodiments of this invention utilize one or more additional additives or reagents (as further described below), the metal complexes useful in this invention may form bridged species with a Group 13 (of the Periodic Table) metal reagent, or divalent or alkali metal reagents as defined herein. Optionally, this complex may be charged and may be associated with a suitable counter ion.

In connection with the metal complex and depending on the ligand or ligands chosen, the metal complex may take the form of dimers, trimers, or higher orders thereof, or there may be two or more metal atoms that are bridged by one or more ligands.

Activators/Additives/Modifiers

In some embodiments, the resulting metal-ligand composition may be directly screened for reactivity in a chemical reaction of interest. In other embodiments, however, the resulting metal-ligand combinations generates active catalysts in combination with a suitable additive, such as an activator, or some other activating technique. In still other embodiments, as further described herein, the resulting metal-ligand compound or combination may first be combined with one or more reactants for the screening reaction of interest, and then an activator will be added.

Generally speaking, suitable activators for use in the present process include alumoxanes, Lewis acids, Bronsted acids, compatible non-interfering activators and combinations of the foregoing. These activators have been previously disclosed for use with different compositions or metal complexes in the following references, which are hereby incorporated by reference in their entirety: U.S. Pat. Nos. 5,599,761, 5,616,664, 5,453,410, 5,153,157, 5,064,802, and European Patent No. EP-A-277,004.

In some embodiments, ionic or ion forming activators may be employed and, in some instances, are preferred. Suitable ion forming compounds useful as an activator in some embodiments of the present invention comprise a cation which is a Bronsted acid capable of donating a proton and an inert, compatible, non-interfering anion, A⁻. Preferred anions are those containing a single coordination complex comprising a charge-bearing metal or metalloid core. Mechanistically, in one embodiment the anion should be sufficiently labile to be displaced by olefinic, diolefinic and acetylenically unsaturated compounds or other neutral Lewis bases such as ethers or nitrites. Suitable metals include, but are not limited to, aluminum, gold and platinum. Suitable metalloids include, but are not limited to, boron, phosphorus, and silicon. Compounds containing anions that comprise coordination complexes containing a single metal or metalloid atom are well known and many, particularly such compounds containing a single boron atom in the anion portion, are available commercially.

In a favored embodiment, these ion forming activators may be represented by the following general formula:

(L*—H)_{d}⁺(A^{d-})

wherein: L* is a neutral Lewis base; (L*—H)⁺is a Bronsted acid; A^{d-}is a non-interfering, compatible anion having a negative charge of d, d ranging from about 1 to 3. More preferably A^{d-}corresponds to the formula:

[M′³⁺Qₕ]^{d-}

wherein: h ranges from about 4 to 6; d=h−3 (d typically ranging from about 1 to 3); M′ is an element selected from Group 13 (of the Periodic Table of the Elements); and, Q is independently selected from the group consisting of hydride, dialkylamido, halide, alkoxide, aryloxide, hydrocarbyl, and substituted-hydrocarbyl radicals (including halosubstituted hydrocarbyl, such as perhalogenated hydrocarbyl radicals), Q typically having up to about 20 carbons. In a more preferred embodiment, d is one; that is, the counter ion has a single negative charge and corresponds to the formula A⁻.

Activators comprising boron or aluminum which are particularly useful in some embodiments of this invention may be represented by the following general formula:

[L*—H]⁺[JQ₄]⁻

wherein: L* is as previously defined; J is boron or aluminum; and Q is a fluorinated C₁₋₂₀hydrocarbyl group. Most preferably, Q is independently selected from the group consisting of fluorinated aryl groups, especially a pentafluorophenyl group (i.e., a C₆F₅ group), or a 3, 5-(CF₃)₂C₆H₃ group. Illustrative, but not limiting, examples of boron compounds which may be used as an activating cocatalyst in the preparation of the metal-ligand compositions of this invention include: (i) tri-substituted ammonium salts such as: trimethylammonium tetraphenylborate, triethylammonium tetraphenylborate, tripropylammonium tetraphenylborate, tri(n-butyl)ammonium tetraphenylborate, tri(t-butyl)ammonium tetraphenylborate, N,N-dimethylanilinium tetraphenylborate, N,N-diethylanilinium tetraphenylborate, N,N-dimethylanilinium tetra- (3,5-bis(trifluoromethyl)phenyl)borate, N,N-dimethyl-(2,4,6-trimethylanilinium) tetraphenylborate, trimethylammonium tetrakis(pentafluorophenyl) borate, triethylammonium tetrakis(pentafluorophenyl) borate, tripropylammonium tetrakis(pentafluorophenyl) borate, tri(n-butyl)ammonium tetrakis(pentafluorophenyl) borate, tri(secbutyl)ammonium tetrakis(pentafluorophenyl) borate, N,N-dimethylanilinium tetrakis(pentafluorophenyl) borate, N,N-diethylanilinium tetrakis(pentafluorophenyl) borate, N,N-dimethyl-(2,4,6-trimethylanilinium) tetrakis(pentafluorophenyl) borate, trimethylammonium tetrakis- (2,3,4, 6-tetrafluorophenylborate and N,N-dimethylanilinium tetrakis-(2,3,4,6-tetrafluorophenyl) borate; (ii) dialkyl ammonium salts such as: di-(i-propyl)ammonium tetrakis(pentafluorophenyl) borate, and dicyclohexylammonium tetrakis(pentafluorophenyl) borate; and, (iii) tri-substituted phosphonium salts such as: triphenylphospnonium tetrakis(pentafluorophenyl) borate, tri(o-tolyl)phosphonium tetrakis(pentafluorophenyl) borate, tri(2,6-dimethylphenyl)phosphonium tetrakis-(pentafluorophenyl) borate and N,N-dimethylanilinium tetrakis(3,5-bis(trifluoromethyl)phenyl)borate. Preferred [L*-H]⁺cations include N,N-dimethylanilinium and tributylammonium. Preferred anions include tetrakis(3,5-bis(trifluoromethyl)phenyl)borate and tetrakis-(pentafluorophenyl)borate. In some embodiments, the most preferred activator is PhNMe₂H⁺B(C₆F₅)₄⁻.

Other suitable ion-forming activators comprise a salt of a cationic oxidizing agent and a non-interfering, compatible anion represented by the formula:

(OX^{e+})_{d}(A^{d-})ₑ

wherein: Ox^{e+}is a cationic oxidizing agent having a charge of e+, e ranging from about 1 to 3; and, A^{d-}and d are as previously defined. Examples of cationic oxidizing agents include: ferrocenium, hydrocarbyl-substituted ferrocenium, Ag⁺or Pb⁺². Preferred embodiments of A^{d-} are those anions previously defined with respect to the Bronsted acid-containing activating cocatalysts, especially tetrakis-(pentafluorophenyl)borate.

Another preferred embodiment of suitable ion forming, activating cocatalysts comprises a compound which is a salt of a carbenium ion or silyl cation and a non-interfering, compatible anion represented by the formula:

^{©+}A⁻

wherein: ^{©+}is a C₁₋₁₀₀ carbenium ion or silyl cation; and, A⁻ is as previously defined. A preferred carbenium ion is the trityl cation (i.e. triphenylcarbenium). The silyl cation may be characterized by the formula Z¹Z²Z³Si⁺, where each of Z¹, Z² and Z³ is independently selected from the group consisting of alkyl, substituted alkyl, cycloalkyl, heterocycloalkyl, heterocyclic, aryl, substituted aryl, heteroaryl, substituted heteroaryl and combinations thereof. In some embodiments, a most preferred activator is Ph₃C⁺B (C₆F₅)₄⁻.

In addition to the foregoing, suitable activators may also include Lewis acids, such as those selected from the group consisting of tris(aryl)boranes, tris(substituted aryl)boranes, tris(aryl)alanes, tris(substituted aryl)alanes, including activators such as tris-(pentafluorophenyl)borane. Other useful ion-forming Lewis acids include those having two or more Lewis acidic sites, such as those described in PCT Application No. WO 99/06413 or those described by Piers et al., “New Bifunctional Perfluoroaryl Boranes: Synthesis and Reactivity of the ortho-Phenylene-Bridged Diboranes 1,2−[B(C₆F₅)₂]₂C₆X₄ (X=H, F),” J. Am. Chem. Soc., 1999, 121, 3244-3245, both of which are incorporated herein by reference. Other useful Lewis acids will be evident to those of skill in the art.

Other general activators or compounds useful in a catalytic reaction, for example in a polymerization reaction, may be used. These compounds may be activators in some contexts, but may also serve other functions in the catalytic system, such as alkylating a metal center or scavenging impurities. These compounds include Group 13 reagents that may be characterized by the formula:

G¹³R′₃₋ₚDₚ

wherein: G¹³ is a Group 13 element selected from the group consisting of Al, B, Ga, In and combinations thereof; p is about 0, 1 or 2; each R′ is independently selected from the group consisting of alkyl, substituted alkyl, cycloalkyl, heterocycloalkyl, aryl, substituted aryl, heterocyclic and combinations thereof; and, each D is independently selected from the group consisting of halide, hydride, alkoxy, aryloxy, amino, thio, phosphino and combinations thereof. Examples of such compounds include a tri-alkyl aluminum, specifically trimethylaluminum, triethylaluminum, or triisobutylaluminum, as well as di-alkyl aluminum hydride, such as di-isobutyl aluminum hydride. The Group 13 activator may also be an oligomeric or polymeric alumoxane compound, such as methylalumoxane and the known modifications thereof.

In still other embodiments, a divalent metal reagent may be used that is defined by the general formula:

M′R′₂₋ₚ,Dₚ,

wherein: p′ is about 0 or 1; R′ and D are as defined above; and, M′ is a metal selected from the group consisting of Mg, Ca, Sr, Ba, Zn, Cd and combinations thereof.

In still other embodiments, an alkali metal reagent may be used that is defined by the general formula:

M″R′

wherein: R′ is as defined above; and, M″ is an alkali metal selected from the group consisting of Li, Na, K, Rb, Cs and combinations thereof.

Additionally, hydrogen and/or silanes may be used in the catalytic composition or added to the catalytic system. Silanes may be characterized by the formula:

SiR′_{4-q}D_{q}

where: R′ i as defined above; q is about 1, 2, 3 or 4; and, D is as defined above, with the proviso that at least one D is a hydride.

The molar ratio of metal precursor to activator employed preferably ranges from about 1:10,000 to about 100:1, more preferably from about 1:8000 to about 80:1, more preferably from about 1:7000 to about 60:1, more preferably from about 1:6000 to about 40:1, more preferably from about 1:5000 to about 10:1, more preferably from about 1:2500 to about 8:1, more preferably from about 1:1000 to about 6:1, more preferably from about 1:500 to about 4:1, more preferably from about 1:100 to about 2:1, and most preferably from about 1:10 to about 1:1. In a preferred embodiment of the invention, mixtures of the above compounds are used, particularly a combination with a Group 13 reagent (i.e., a composition or complex wherein one or more of the metals is a Group 13 element) and an ion-forming activator. The molar ratio of Group 13 reagent to ion-forming activator is preferably from about 1:10,000 to about 1000:1, more preferably from about 1:7500 to about 500:1, more preferably from about 1:5000 to about 100:1, most preferably from about 1:100 to about 100:1 (e.g., from about 1:75 to 75:1, about 1:50 to about 50:1, or about 1:25 to about 25:1). In a preferred embodiment, the ion forming activators are combined with a tri-alkyl aluminum (more specifically a trimethylaluminum, triethylaluminum or triisobutylaluminum), or with a di-alkyl aluminum hydride (such as a di-isobutyl aluminum hydride).

Combinatorial Methodoloqv

The metal-ligand compositions or compounds of the present invention can be prepared, and then tested or screened, such as for reactivity or, more specifically, catalytic activity for example, in one or more reactions of interest (as further described herein) in a combinatorial fashion. Combinatorial chemistry generally involves the parallel or rapid serial synthesis and/or screening or characterization of compounds and compositions of matter. (See, e.g., U.S. Pat. No. 5,985,356; U.S. Pat. No. 6,030,917; PCT Application No. WO 98/03521; and, U.S. patent application Ser. No. 09/227,558, filed on Jan. 8, 1999, all of which are incorporated herein by reference and which generally disclose combinatorial methods.)

In this approach, the soluble metal precursors, ligands, metal-ligand complexes or compositions may be prepared and/or tested in rapid serial and/or parallel fashion (e.g., in an array format). When prepared in an array format, for example, the soluble metal precursors, activators and/or ligands may take the form of an array comprising a plurality of compounds wherein each compound can be characterized by the general formulas described above. Typically, each member of the array will have differences so that, for example, a ligand or activator or R group or metal in a first region of the array may be different than the ligand or activator or R group or metal in a second region of the array. Other variables may also differ from region to region in the array.

In such a combinatorial array, typically each of the plurality of compositions or complexes has a different composition or stoichiometry, and typically each composition or complex is at a selected region on a substrate such that each compound is isolated from the other compositions or complexes. This isolation can take many forms, typically depending on the substrate used. If a flat substrate is used, there may simply be sufficient space between regions so that there cannot be interdiffusion between compositions or complexes. As another example, the substrate can be a microtiter or similar plate having wells so that each composition or complex is in a region separated from other compounds in other regions by a physical barrier. The array may also comprise a parallel reactor or testing chamber.

Generally speaking, in accordance with the process of the present invention, one or more metal-solublizing ligand compounds or complexes are formed (examples of which are further described herein). Once formed, these soluble metal precursors may be dissolved in a suitable solvent and then transferred to discrete regions of a substrate (as described above), where it may be combined with another metal-binding ligand, an activator, some other additive, or some combination thereof, prior to screening. Alternatively, the soluble metal precursor may be screened directly for reactivity of some kind.

Solvent selection for the soluble metal precursors may vary. Preferably, however, the solvent used to dissolve the metal precursor for delivery to the array will be the same solvent in which the screening reaction is to be performed, or a solvent compatible therewith. Additionally, the solvent in which the soluble metal precursor is dissolved/delivered will also be solublizing to the member ligand in the array; that is, in some embodiments it is preferable for the ligand to be dissolved by the same solvent as the soluble metal precursor. Alternatively, however, the soluble metal precursor may be solubilized and delivered to the array in a solvent which is subsequently removed prior to the screening reaction.

Solvents typically preferred for use in one or more embodiments of the present process include, for example, hydrocarbon solvents (including aliphatic hyrdrocarbons, for example pentanes, hexanes, pentanes, heptanes, octenes, Isopar-E, as well as aromatic hyrdrocarbons, for example toluene, bexzene, xylene and mesitylene), clorinated solvents (for example, methylene chloride, 1,2-dichloroethane (“DCE”), chlorobenzene, diclhlorobenzene, and trichlorobenzene). Other solvents which may be used include ether solvents (for example, diethylether, tetrahydrofuran (“THF”),dimethoxyethane (“DME”), and diisopropylether), as well as other solvents like acetonitrile.

It is to be noted that, in some embodiments, preferably a non-protic solvent will be employed, and more preferably the entire screening medium will be non-protic. As used herein, screening medium generally refers to the contents of the well of the array just prior to the screening reaction of interest being performed. Additionally, a “non-protic” solvent or medium generally refers to a solvent and/or medium which is substantially free of a protic liquid (e.g., less than about 1 part in 1000, 1 part in 750, 1 part in 500, or less).

It is to be further noted that, for olefin polymerization, preferred solvents are typically hydrocarbon solvents or chlorinated solvents, the most preferred solvents being hydrocarbon solvents.

The array which is formed typically comprises at least about 8 compounds, complexes or compositions, each having a different chemical formula (meaning that there must be at least one different atom or bond differentiating the members in the array or different ratios of the components referred to herein, with components referring to soluble metal precursors, activators, Group 13 reagents, solvents, monomers, supports, etc.). In other embodiments, there are at least about 15, 20, 23 or even 40 compounds, complexes or compositions on or in the substrate, each having a different chemical formula. In still other embodiments, there are at least about 50, 80, 100, 200, 300, 500, 1000, 10000 or even 10⁶ compounds, complexes or compositions on or in the substrate (each having a different chemical formula as described above). Because of the manner of forming combinatorial arrays, it may be that each compound, complex or composition may not be worked-up, purified or isolated and, for example, may contain reaction by-products or impurities or unreacted starting materials.

Once formed, the metal-ligand compositions may be subjected to one or more screening reactions of interest. Such reactions include, for example, oxidation, reduction, hydrogenation, hydrosilylation, hydrocyanation, hydroformylation, polymerization, carbonylation, isomerization, metathesis, carbon-hydrogen activation, carbon-halogen activation, cross-coupling, Friedel-Crafts acylation and alkylation, hydration, dimerization, trimerization, oligomerization, and Diels-Alder reactions, among other transformations.

Of particular interest in some embodiments is the screening of compounds for catalytic activity in polymerization or copolymerization reactions. The catalytic performance of the compounds, complexes or compositions of this invention can be tested in a combinatorial or high throughput fashion. Polymerizations can also be performed in a combinatorial fashion (see, e.g., U.S. patent application Ser. Nos. 09/211,982, filed Dec. 14, 1998 and 09/239,223, filed Jan. 29, 1999, each of which is herein incorporated by reference).

The compositions and catalysts generated in accordance with the present process may generally be used, for example, to oligomerize or polymerize olefinically or acetylenically unsaturated monomers having from about 2 to about 20 carbon atoms either alone or in combination. The compounds and catalysts of this invention may also be used to polymerize functionalized monomers. Monomers which may be polymerized include, for example: olefins, diolefins, and acetylenically unsaturated monomers (including ethylene and C₃ to C₂₀ α-olefins, such as propylene, 1-butene, 1-hexene, 1-octene, 4-methyl-l-pentene, 1-norbornene, styrene and mixtures thereof); additionally, 1,1-disubstituted olefins, such as isobutylene, either alone or with other monomers, such as ethylene or C₃ to C₂₀ α-olefins and/or diolefins, may also be used.

It is to be noted that these definitions are intended to include cyclic olefins, as well. Furthermore, vinyl monomers with functional groups may also be polymerized alone (e.g., in a homopolymerization) or with other monomers (such as ethylene or C₃ to C₂₀ α-olefins). Such functional group containing vinyl monomers can be characterized by the general formula H₂C=CH-FG, where FG is the functional group that contains at least one heteroatom (as defined herein) or halogen (e.g., Cl, F, Br, etc.). Functional monomers include C₁ to C₂₀ acrylates, C₁ to C₂₀ methacrylates, acrylic acid, methacrylic acid, maleic anhydride, vinyl acetate, vinyl ethers, acrylonitrile, acrylamide, vinyl chloride and mixtures thereof.

It is to be noted that once the screening reactions have been performed, one or more reaction products, such as polymerization reaction products (including an oligomer, such as a dimer, trimer, etc., as well as a polymer), may result. The process of the present invention may therefore additionally comprise the screening or testing of one or more of the reaction products in this product array for a property of interest including, for example, electrical, thermal, mechanical, morphological, optical, magnetic or chemical properties. These properties may be evaluated by subjecting the reaction product to, for example, infrared spectroscopy, infrared imaging, a parallel polymerization reaction, liquid chromatography, light scattering, polymer structural testing, polymer melt flow testing, polymer property testing, Fourier Transform Infrared spectroscopy, Raman spectroscopy, thin layer chromatography, solid phase staining, rapid gel permeation chromatography, nuclear magnetic resonance spectroscopy, depolarized light scattering, rapid thermal analysis, gas composition-mass spectrometry, X-ray fluorescence spectroscopy, and/or liquid composition-mass spectrometry.

The process of the present invention is further illustrated by the following Examples. The Examples are designed to teach those of ordinary skill in the art how to practice the process of the invention. Accordingly, these Examples are not to be interpreted in a limiting sense.

### EXAMPLES

As further described below, the following Examples illustrate various feature of the present invention. More specifically, Examples 1 through 41 illustrate the process of the present invention, wherein a solution comprising a dissolved, soluble metal precursor is delivered to one or more member ligands of a ligand array to prepare various metal-ligand compositions, or compounds, which may then be screened in one or more reactions of interest. Examples 42 through 49 illustrate the preparation of particularly preferred, novel soluble metal precursors.

### Examples 1 through 41

All procedures were performed in an inert atmosphere dry box, under an atmosphere of purified argon or nitrogen, and standard purification methods were used to dry and deoxygenate the solvents and reagents, as would be known to one skilled in the art of olefin polymerization using organometallic catalysts. All glassware (vials, pipettes, tubes, bottles, reservoirs, syringes, etc.) was dried in an oven at 120° C. for at least 2 hours and placed under vacuum for at least 2 hours prior to use in experiments. All plastics (vials, vial caps, lids, pipette tips, reservoirs, troughs, microtiter plates, syringes, etc.) were dried in a vacuum oven at 60-80° C. for 2-3 hours and then further dried under vacuum for at least 12 hours prior to use. Polymer Grade ethylene from Matheson was used in screening and was further purified by passing it through a Matheson Model 6427-4S Oxygen/Moisture drying tube before use. All synthetic manipulations and screening of libraries involved the use of standard 96-well polypropylene microtiter plates.

General Synthetic Protocols-Ligand Arrays: The substrate employed in this invention consists of a 96-well polypropylene microtiter plate with 8 rows of 12 columns. The rows are labelled from A to H and the columns from 1 to 12. To each well of the array is optionally added 3.0×10⁻⁴ mmol of a ligand. In Examples 1-17, the 96-element array is divided into 4 equivalent zones, each consisting of three columns of eight wells, where 21 of the 24 wells contained ligands (21 different ligands). The 21 different ligands were chosen from the classes of ligands described as being useful in this invention in the specification. In Examples 18-38, the 96-element array is divided into 4 equivalent zones, each consisting of three columns of eight wells, where 19 of the 24 wells contained ligands (19 different ligands). The 19 different ligands were chosen from the classes of ligands described as being useful in this invention in the specification. In Examples 39-41, a single zone contain about 90 well was employed.

General Synthetic Protocols-Synthetic Manipulations: Synthetic manipulations in an array format were performed by preparing stock solutions of the reagents (reagents include but are not limited to solvents, ligands, soluble metal precursors, metal-ligand complexes and/or combinations, activators, scavengers, co-catalysts, monomers and other components typically used in reactions of this type) and transferring them to a microtiter plate using a single or multichannel pipettor or a liquid handling robot. Solutions were prepared and optionally transferred into Teflon, polypropylene, or glass troughs or vials, and then subsequently transferred to the microtiter plate array. Once the reagents were added the contents of the wells (see Table I, below) each of the arrays were shaken using an orbital shaker for about 3-5 minutes. The contents of the wells of the arrays were then allowed to react for a desired length of time. The resulting reaction products were then ready for further synthetic manipulation or immediate activation and use in a screening experiment (as detailed in Table I, below).

Examples Employing Soluble Metal Precursors: The Examples below illustrate the use of metal-alkyl and/or metal-aryl soluble metal precursors and soluble inorganic metal precursors in the present invention.

Example 1

Preparation of Ligand Array: The ligand array in this example consists of a 96-well microtiter plate with the 4 zone arrangement described above, wherein each zone contains 21 ligands (3.0×10⁻⁴ mmol of ligand per well) and 3 empty wells. The empty wells are A1, A4, A7, A10, Bl, B4, B7, B10, C2, CS, C8, and Cll.

Addition of Soluble Metal Precursor to the Ligand Array: A 4.4×10⁻³ molar solution of a soluble metal precursor was prepared by dissolving 18.2 mg of Ti(CH₂Ph)₄ in 10.0 mL of toluene in a glass vial. The solution of the metal precursor was then transferred to a Teflon trough. Using a multichannel pipettor, 75.0 μL of this solution (3.3 ×10⁻⁴ mmol, 1.1 equivalent per 1 equivalent of ligand) was then transferred to each well of the array except wells A1, A4, A7, A10, C2, C5, C8, and C11. To each of these empty wells was added 75.0 μL of toluene and the array was then shaken on a vortexing mixer for 3-5 minutes. The plate was then allowed to stand for 1 hour at room temperature. The array was then exposed to ethylene at 60 psi, activated and the heat of reaction was monitored using infrared thermography for a period of one hour.

Examples 2-17

Examples 2-17 were performed following the same procedure as that described for Example 1, the metal precursor and “standing time” at room temperature prior to reaction being changed as indicated in Table 1, below.

Example 18

Preparation of Ligand Array: The ligand array in this example consists of a 96-well microtiter plate with the 4 zone arrangement described above, wherein each zone contains 19 ligands (3.0×10⁻⁴ mmol of ligand per well) and 5 empty wells. The empty wells are A1, A4, A7, A10, B2, B5, B8, B11, E2, E5, E8, E11, G1, G4, G7, G10, G3, G6, G9 and G12.

Addition of Soluble Metal Precursor to the Ligand Array: A 4.4×10⁻³ molar solution of the soluble metal precursor was prepared by dissolving 25.9 mg of [Fe(mesityl)₂]₂ in 20 mL of toluene in a glass vial. The solution of the metal precursor was then transferred to a Teflon trough. Using a multichannel pipettor, 75.0 μL of this solution (3.3×10⁻⁴ mmol, 1.1 equivalent per 1 equivalent of ligand) was then transferred to each well of the array except wells A1, A4, A7, A10, E2, E5, E8 and E11. To each of these empty wells was added 75.0 μL of toluene and the array was then shaken on a vortexing mixer for 3-5 minutes. The plate was then allowed to stand for 10 minutes. The array was then exposed to ethylene at 60 psi, activated and the heat of reaction was monitored using infrared thermography for a period of one hour.

Examples 19-35

Examples 19-35 were performed following the same procedure as that described for Example 18, the metal precursor being changed as indicated in Table 1, below.

Examples 36-37

Preparation of Ligand Array: The ligand array in this example was prepared following the procedure described in Example 18.

Addition of Soluble Metal Precursor to the Ligand Array: A 4.4×10⁻³ molar solution of three different soluble metal precursors, “M(TFA)₂” (where M=Fe, Co and Ni for zones 1, 2 and 3, respectively), was prepared using Et₂O/DCE as a solvent (2% Et₂O by volume), and one soluble metal precursor, “M(acac)₂” (where M=Ni for zone 4), was prepared in toluene. Using a multichannel pipettor, about 75.0 μL of each solution (3.3×10⁻⁴ mmol, 1.1 equivalents per 1 equivalent of ligand) was transferred to each well of the array. After mixing, contents of the wells of the array were allowed to react for four hours at room temperature. Solvent was then removed and the plate was dried in vacuo. About 100 μL toluene was then added to each well and mixed on shaker plate. The plate was then pressurized with ethylene (60 psi). The heat of reaction was monitored using infrared thermography for a period of one hour.

Example 38

Preparation of Ligand Array: A 90-member library of neutral ligands was assembled in a microtiter plate using a protocol similar to that described for Example 1.

Addition of Soluble Metal Precursor to the Ligand Array: Aliquots (75 μL) of 4.4×10⁻³ molar solutions in chlorobenzene of soluble metal precursors CODPdMeOTf, CODPdMeTFA and CODPdMeCl were added to zones 1, 2 and 3-4, respectively (3.3×10⁻⁴ mmol, 1.1 equivalent per 1 equivalent of ligand). The plate was then mixed for 10 minutes at room temperature. The array was then exposed to ethylene at 60 psi, activated and the heat of reaction was monitored using infrared thermography for a period of one hour.

Example 39:

Preparation of Ligand Array: A 90-member library of neutral ligands was assembled in a microtiter plate using a protocol similar to that described for Example 1.

Addition of a Modifier to the Ligand Array: A solution of 1.2×10⁻² molar Ag(BAr_{F})₄ in 1,2-dichloroethane was prepared by dissolving 232.8 mg Ag(BAr_{F})₄ in 20 mL of 1,2-dichloroethane toluene. A 25 μL aliquot of this solution was then added to each well of the array of ligands via multichannel pipettor and the volatile components of each well in the array were allowed to evaporate through the assistance of a stream of nitrogen directed over each well. When the contents of the wells in the array were dry the array was further dried in vacuo for a few hours.

Addition of Soluble Metal Precursor to the Ligand Array: A 3.0×10⁻³ molar solution of a soluble metal precursor was prepared by dissolving 42 mg of (PPh₃)₂Ni(Ph)Cl in 20.1 mL of toluene in a glass vial. The solution of the metal precursor was then transferred to a Teflon trough. Using a multichannel pipettor, 100 AL of this solution (3.0×10⁻⁴ mmol, 1.0 equivalent per 1 equivalent of ligand) was then transferred to selected wells in the array. To each of the these empty wells was added 100 μL of toluene and the array was then thoroughly agitated and allowed to react for 30 minutes. The array was then exposed to ethylene at 60 psi, activated and the heat of reaction was monitored using infrared thermography for a period of one hour.

Example 40

Preparation of Ligand Array: A 90-member library of neutral ligands was assembled in a microtiter plate using a protocol similar to that described for Example 1.

Addition of Soluble Metal Precursor to the Ligand Array: A 3.6×10⁻³ molar solution of (py)₂Ni(CH₂SiMe₃)₂ in toluene was prepared by dissolving 28 mg of (py)₂Ni(CH₂SiMe₃)₂ in 20 mL of toluene in a glass vial. Using a multichannel pipettor, 100 μL of this solution (3.6×10⁻⁴ mmol, 1.2 equivalent per 1 equivalent of ligand) was then transferred to selected wells in the array. To each of the these empty wells was added 100 μL of toluene. The array was thoroughly agitated and allowed to react for 15 minutes, and then allowed to further react for 13 hours at room temperature without agitation. The volatiles were allowed to evaporate and then the plate was further dried for 2 hours under vacuum. Toluene (100 μL) was added to each well and the plate was agitated for 30 minutes. The array was then exposed to ethylene at 60 psi, activated and the heat of reaction was monitored using infrared thermography for a period of one hour.

Example 41

Preparation of Ligand Array: A 45-member library of charged ligands was assembled in a microtiter plate was assembled using a protocol simlar to that described for Example 1.

Addition of Soluble Metal Precursor to the Ligand Array: A 3.6×10⁻³ molar solution of (py)₂Ni(CH₂SiMe₃)₂ in toluene was prepared by dissolving 28 mg of (py)₂Ni(CH₂SiMe₃)₂ in 20 mL of toluene in a glass vial. Using a multichannel pipettor, 100 μL of this solution (3.6×10⁻⁴ mmol, 1.2 equivalent per 1 equivalent of ligand) was then transferred to selected wells in the array. To each of the these empty wells was added 100 μL of toluene. The array was thoroughly agitated and allowed to react for 25 minutes, and then allowed to further react for 12 hours at room temperature without agitation. The volatiles were allowed to evaporate and then the plate was further dried for 2 hours under vacuum. Toluene (125 μL) was added to each well and the plate was agitated for 25 minutes. The array was then exposed to ethylene at 60 psi, activated and the heat of reaction was monitored using infrared thermography for a period of one hour.

### Examples 42 through 49

Examples 42 through 49 detail the preparation of soluble metal precursors suitable for use in the present process.

Example 42

Preparation of (COD)PdMe(TFA): Solid silver trifluoroacetate (224 mg, 1.02 mmol) and CODPdMeCl (270 mg, 1.02 mmol) were combined and 10 mL dichloromethane was added. A colorless precipitate formed and the resulting mixture was allowed to stir for 3 hours. The mixture was filtered. The colorless filtrate was then evaporated, yielding a white solid which was washed with 10 mL pentane and dried. (314 mg; 94%) ¹H NMR (C₆D₆) 5.53 (br s, 2, COD olefinic), 4.05 (br s, COD olefinic), 1.60, 1.42 (m, 4 each, CH=CHCH₂), 1.02 (s, 3, PdCH₃).

Example 43

Preparation of (p-cymene) Ru (TFA)₂ (CH₃CN): Solid [(p-cymene)RuCl₂]₂ (3.17 g, 5.18 mmol) and AgTFA (4.61 g, 20.86 mmol) were combined. CH₂Cl₂ (100 mL) and CH₃CN (3 mL) were added and the mixture was stirred for 2.5 hours. The reaction mixture was filtered through celite, all solids were washed well with CH₂Cl₂, and solvent was removed from the filtrate in vacuo. A yellow-orange solid was obtained which was washed with Et₂O (50 mL) and pentane (100 mL), and then dried (4.02 g, 81%). ¹H NMR was consistent with the title compound.

Example 44

Preparation of (p-cymene)Ru(TFA)(mesityl): (p-cymene)Ru(TFA)₂(CH₃CN) (1009 mg, 2.0 mmol) was dissolved in 20 mL THF. (mesityl)₂Mg(THF)₂ (440 mg, 1.09 mmol) was dissolved in 10 mL THF. Both solutions were cooled to −35° C. and then the Mg(mesityl)₂(THF)₂ solution was added drop-wise to the solution containing the (p-cymene)Ru(TFA)₂(CH₃CN). The resulting red solution was allowed to warm to room temperature and then was stirred for 3 hours. Solvent was removed in vacuo, and the product was extracted with 50 mL Et₂O. The solution was concentrated to 10 mL and refiltered to removed a small amount of a colorless precipitate. Solvent was removed in vacuo from the filtrate, yielding a red oily solid, the ¹H NMR results of which were consistent with the title compound. (477 mg, 52.4%). The compound can optionally be recrystallized from Et₂O/pentane.

Example 45

Preparation of Co[(C(SiMe₃)₃]₂: CoCl₂ (422 mg, 3.27 mmol) was slurried in 20 mL THF and the mixture was cooled to −35° C. [(Me₃Si)₃CLi(THF)₂] (2.39 g, 6.25 mmol) was dissolved in 20 mL THF. The mixture was cooled to −35° C. and then added drop-wise to the cooled slurry of CoCl₂. The resulting dark green mixture was allowed to warm to room temperature and was stirred for 3 hours. Solvent was removed in vacuo, yielding a green oil which was extracted with 20 mL Et₂O, filtered and concentrated. Upon cooling, dark green plate-like crystals were obtained.

Example 46

Preparation of Fe[(C(SiMe₃)₃]₂: Using a procedure similar to that described for Co[(C(SiMe₃)₃]₂, Fe[(C(SiMe₃)₃]₂ was prepared from FeCl₂ (245 mg, 1.93 mmol) and [(Me₃Si)₃CLi(THF)₂] (1. 65 g, 4.32 mmol). An orange-red solid was obtained which was extracted with pentane (20 mL), filtered and concentrated to 2 mL. Upon cooling the solution to −35° C., orange blocks were obtained. The identity of this complex was solved by X-ray diffraction.

Example 47

Preparation of Cr[(C(SiMe₃)₃]₂: Using a procedure similar to that described for Co[(C(SiMe₂)₃]₂, Cr[(C(SiMe₃)₃]₂ was prepared from CrCl₂ and [(Me₃Si)₃CLi(THF)₂].

Example 48

Preparation of [Fe(CH₂SiMe₃)₄]²⁻[Li₂(Et₂O)n]²⁺ (where n ranges from 4 to 8): FeCl₂ (260 mg, 2.04 mmol) was slurried in 7 mL Et20 and then the slurry was cooled to −35° C. A solution of Me₃SiCH₂Li in pentane (9.0 mmol) was added to the cooled slurry. The mixture was allowed to warm to room temperature. The mixture became greenish-brown and a small amount of colorless precipitate formed. The mixture was stirred at room temperature for 2.0 hours. Solvent was removed in vacuo, yielding a paramagnetic orange-red oil which resisted all attempts at recrystallization.

Example 49

Preparation of [Fe(CH₂SiMe₃)₄]²⁻[Li₂(12-crown-4)2]²⁺(where n ranges from 4 to 8): [Fe(CH₂SiMe₃)₄]²⁻[Li₂(Et₂O)n]²⁺ (320 mg) was dissolved in 3 mL Et₂O. 12-crown-4 (230 mg, 1.31 mmol) was dissolved in 1 mL Et₂O and this solution was layered on top of the first solution. The mixture was stirred at room temperature for 1 hour. The mixture was filtered and concentrated to 2 mL. One mL of pentane was then layered on top of the concentrate. Paramagnetic pale yellow needle-like crystals formed and were collected and dried. (253 mg)

In view of the above, it will be seen that the several objects of the invention are achieved. As various changes could be made in the above-described process without departing from the scope of the present invention, it is intended that all matter contained in the above description shall be interpreted as illustrative and not in a limiting sense.

It is to be noted, however, that the polymerization process described herein are not intended to describe the polymerization of nucleic acids or polypetides, proteins and the like.

What is claimed is:

1. A process for preparing and screening an array of metal-ligand compositions comprising:
preparing an array of polymerization mixtures in a series of discrete reaction vessels contained by or within an integrated structure, each polymerization mixture of the array comprising a polymerization monomer and a metal-ligand composition wherein different reaction vessels of the array contain different metal-ligand compositions and said preparing comprises delivering a metal-binding ligand and a dissolved, soluble metal precursor to each of the reaction vessels of the array which combine to form the metal-ligand composition;
subjecting the integrated structure to conditions conducive to the formation of a polymerization reaction product; and
screening the array for polymerization reaction product.

2. The process of claim 1 wherein the metal-ligand compositions comprise a cyclic structure having at least 5 ring members, one member being the metal of the soluble metal precursor.

3. The process of claim 1 wherein the metal-binding ligand is not bound to a solid material.

4. The process of claim 3 wherein the metal-binding ligand is delivered to the series of discrete reaction vessels after the soluble metal precursor.

5. The process of claim 3 wherein the soluble metal precursor is delivered to the series of discrete reaction vessels after the metal-binding ligand.

6. The process of claim 1 wherein the soluble metal precursor has a formula MRₙ, wherein M is a transition metal, a lanthanide metal, an actinide metal, an alkali metal, an alkaline earth metal or a main group metal, R is a solublizing ligand, and n ranges from about 1 to about 8.

7. The process of claim 1 wherein the soluble metal precursor has a formula MRₙL_{m,} wherein M is a transition metal, a lanthanide metal, an actinide metal, an alkali metal, an alkaline earth metal or a main group metal, R is a solublizing ligand, L is a neutral solublizing ligand, n ranges from about 0 to about 8, and m ranges from about 0 to about 6, provided the sum of n+m is at least 1.

8. The process of claim 1 wherein the soluble metal precursor has a formula MₚR_{n′}L_{m′,} wherein M is a transition metal, a lanthanide metal, an actinide metal, an alkali metal, an alkaline earth metal or a main group metal, R is a solublizing ligand, L is a neutral solublizing ligand, p is greater than 1, n′ and m′ are each independently a number greater than 0, provided the sum of n′+m″ is at least 2.

9. The process of claim 1 wherein the soluble metal precursor comprieses a metal selected from Sc, Ti, V, Cr, Mn, Fe, Co, Ni, Cu, Zn, Y, Zr, Nb, Mo, Ru, Rh, Pd, Ag, Cd, La, Hf, Ta, W, Re, Os, Ir, Pt, Au, Hg, Ce, Pr, Nd, Sm, Eu, Gd, Tb, Dy, Ho, Er, Tm, Yb, Lu, U, Th, B, Al, Ga, Tl, In, Li, Na, K, Rb, Cs, Be, Mg, Ca, Sr, Ba, Ge, Sn, Pb, Sb, Bi or a combination thereof.

10. The process of claim 9 wherein the soluble metal precursor comprises one or more solublizing ligands independently selected from the group consisting of halogen, substituted or unsubstituted alkyl, substituted or unsubstituted cycloalkyl, substituted or unsubstituted cycloalkenyl, substituted or unsubstituted heteroalkyl, substituted or unsubstituted heterocycloalkyl, substituted or unsubstituted aryl, substituted or unsubstituted heteroaryl, alkoxy, aryloxy, hydroxy, boryl, silyl, hydride, thio, oxo, imido, sulfido, seleno, phosphino, amino, carboxylate, 1,3-dionate, oxalate, carbonate, nitrate, sulfate, perchlorate, sulfonate, phosphonate and combinations thereof.

11. The process of claim 1 wherein the soluble metal precursor comprises a solublizing ligand R and/or a neutral solublizing ligand L, and further wherein one or more of the metal-ligand compositions is formed without displacing one or more of the solublizing ligands and/or neutral solublizing ligands.

12. The process of claim 1 wherein the soluble metal precursor comprises a solublizing ligand R and/or a neutral solublizing ligand L, and further wherein formation of one or more of the metal-ligand compositions is accompanied by the displacement of one or more of the solublizing ligands and/or neutral solublizing ligands, said displaced ligand(s) not interfering with or significantly inhibiting the polymerization reaction.

13. The process of claim 12 wherein, prior to polymerization reaction, one or more of the metal-ligand compositions is treated to convert the displaced solublizing ligand, R, and/or the displaced neutral solublizing ligand, L, to a form which does not interfere or significantly inhibit the polymerization reaction.

14. The process of claim 1 wherein, prior to the screening reaction, a reactant and an activator are delivered to the polymerization mixture, the reactant being delivered before the activator.

15. The process of claim 1 wherein, prior to the screening reation, a reactant and an activator are delivered to the polymerization mixture, the reactant being delivered after the activator.

16. The process of claim 1 wherein each metal-binding lihand has a coordination number (CN) independently selected from the group consisting of 1, 2, 3 and 4.

17. The process of claim 1 wherein each metal-binding ligand has a charge independently selected from the group consisting of 0, −1, −2 and −3.

18. The process of claim 1 wherein each metal-binding ligand has a coordination number (CN) which is greater than its respective charge.

19. The process of claim 18 wherein each metal-binding ligand has a coordination number (CN) and a charge independently selected from the group consisting of:

| CN | Charge |
|---|---|
| 2 | −2 |
| 2 | −1 |
| 1 | −1 |
| 2 | 0 |
| 3 | −1 |
| 3 | −2 |
| 3 | 0 |
| 4 | 0 |
| 4 | −1 |
| 4 | −2 |
| 1 | 0. |

20. The process of claim 1 wherein each metal-binding ligand has a coordination number (CN) which is less than its respective charge.

21. The process of claim 1 wherein each metal-binding ligand is independently selected from the group consisting of derivatives of the following:

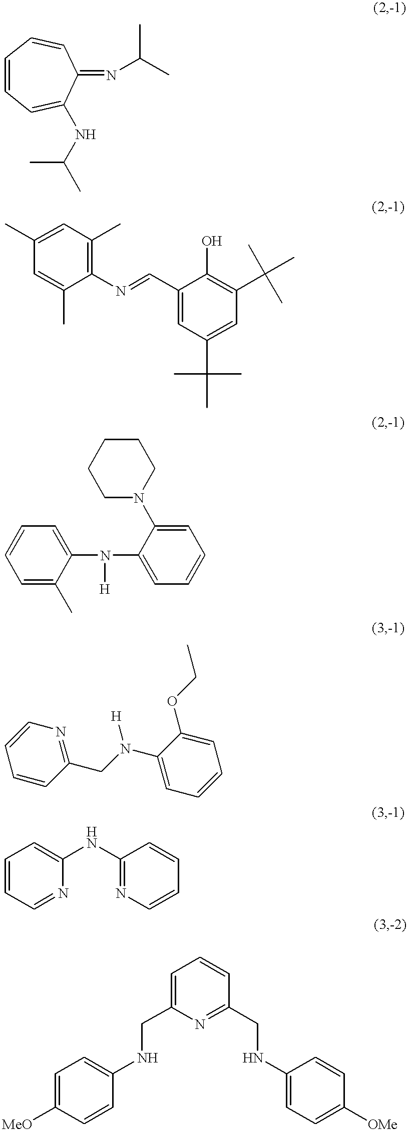

22. The process of claim 1 wherein the soluble metal precursor comprises a substituted alkyl solublizing ligand.

23. The process of claim 22 wherein the solublizing ligand is a trimethylsilyl-substituted alkyl.

24. The process of claim 22 wherein the solublizing ligand is selected from the group consisting of mono(trimethylsilyl)-methyl, bis(trimethylsilyl)methyl and tris(trimethylsilyl)-methyl.

25. The process of claim 1 wherein the soluble metal precursor comprises an alkoxy solublizing ligand.

26. The process of claim 25 wherein the solublizing ligand is tert-butoxy.

27. The process of claim 1 wherein the soluble metal precursor comprises an aryloxy solublizing ligand.

28. The process of claim 27 wherein the solublizing ligand is 2,6-bis(tert-butyl)phenoxy.

29. The process of claim 1 wherein the soluble metal precursor comprises an amino solublizing ligand.

30. The process of claim 29 wherein the solublizing ligand is N,N-bis(trimetylsilyl)amino or N,N-diethylamino.

31. The process of claim 1 wherein the soluble metal precursor comprises a 1,3-dionate solublizing ligand.

32. The process of claim 31 wherein the solublizing ligand is 2,2,6,6-tetramethyl-3,5-heptanedionate.

33. The process of claim 31 wherein the solublizing ligand is 2,4-pentanedionate.

34. The process of claim 1 wherein the soluble metal precursor comprises a carboxylate solublizing ligand.

35. The process of claim 34 wherein the solublizing ligand is trifluoroacetate.

36. The process of claim 1 wherein the soluble metal precursor comprises a substituted or unsubstituted cycloalkenyl solublizing ligand.

37. The process of claim 36 wherein the solublizing ligand is cyclopentadienyl.

38. The process of claim 1 wherein the soluble metal precursor has the formula MRₙLₘ wherein M is a transition metal, a lanthanide metal, an actinide metal, an alkali metal, an alkaline earth metal or a main group metal, R is a solublizing ligand, L is a neutral solublizing ligand, n ranges from about 0 to about 8, and m ranges from about 0 to about 6, provided the sum of n+m is at least 1, and further wherein said soluble metal precursor is negatively charged, and the metal precursor solution additionally comprising a counter cation.

39. The process of claim 1 wherein the soluble metal precursor has the formula LnR₃, wherein R is independently selected from the group consisting of halo, substituted or unsubstituted alkyl, substituted or unsubstituted cycloalkyl, substituted or unsubstituted heteroalkyl, substituted or unsubstituted heterocycloalkyl, substituted or unsubstituted aryl, substituted or unsubstituted heteroaryl, alkoxy, aryloxy, hydroxy, boryl, silyl, hydride, thio, seleno, phosphino, amino, carboxylate, dionate, oxalate, carbonate, nitrate, sulfate, perchlorate, sulfonate, phosphonate and combinations thereof, and wherein Ln is selected from the group consisting of La, Ce, Pr, Nd, Sm, Eu, Gd, Tb, Dy, Ho, Er, Tm, Yb, Lu or a combination thereof.

40. The process of claim 1 wherein the soluble metal precursor comprises more than one solublizing ligand, each being the same.

41. The process of claim 40 wherein the solublizing ligand is selected from the group consisting of halo, hydride, substituted or unsubstituted alkyl, substituted or unsubstituted cyclocalkyl, substituted or unsubstituted aryl, substituted or unsubstituted heteroalkyl, alkoxy, aryloxy, dionate, carboxylate and amino.

42. The process of claim 1 wherein the soluble metal precursor has the formula MRₙLₘ, wherein M is a transition metal, R is a solublizing ligand, L is a neutral solublizing ligand, n ranges from about 0 to 7, m ranges from about 1 to 6, and n+m ranges from about 1 to 7.

43. The process of claim 1 wherein the soluble metal precursor has the formula MRₙLₘ, wherein M is a lanthanide metal, R is a solublizing ligand, L is a neutral solublizing ligand, n ranges from about 0 to 7, m ranges from about 1 to 6, and n+m ranges from about 1 to about 9.

44. The process of claim 1 wherein the soluble metal precursor has the formula MₚR_{n′}L_{m′}, wherein M is a transition metal, R is a solublizing ligand, L is a neutral solublizing ligand, p is at least about 2, n′ ranges from 0 to 7, m′ ranges from 1 to 6, and n′+m′ ranges from about 2 to about 7.

45. The process of claim 1 wherein the soluble metal precursor has the formula MₚR_{n′},L_{m′}, wherein M is a lanthanide metal, R is a solublizing ligand, L is a neutral solublizing ligand, p is at least about 2, n′ ranges from 0 to 7, m′ ranges from 1 to 6, and n′+m′ ranges from about 2 to about 9.

46. The process of claim 1 wherein the soluble metal precursor comprises one or more neutral solublizing ligands, L, independently selected from the group consisting of carbon monoxide, isocyanide, nitrous oxide, alkylnitrile, arylnitrile, PX₃, NX₃, OX₂, SX₂, SeX₂, OPX₃, ONX₃, SPX₃, OSX₂, OSOX₂, X₂N-NX₂, XS-SX, XO-OX, η⁶-arene, olefin, alkenyl, cycloalkenyl, alkynyl and combinations thereof, wherein each X is independently selected from the group consisting of substituted or unsubstituted alkyl, substituted or unsubstituted cycloalkyl, substituted or unsubstituted heteroalkyl, substituted or unsubstituted heterocycloalkyl, substituted or unsubstituted aryl, substituted or unsubstituted heteroaryl, silyl and combinations thereof.

47. The process of claim 1 wherein prior to the polymerization reaction one or more of the polymerization mixtures is activated.

48. The process of claim 47 wherein activation is achieved by the addition of one or more activators independently selected from the group consisting of methylalumoxane, trialkylaluminum, dialkylaluminumhydride, 5 diethylaluminumethoxide, diethylaluminumchloride, dibutylmagnesium, dialkylzinc, trialkylboron, AgBF₄, AgBPh₄, NaBAr₄, AgBAr₄, [H(OEt₂)]⁺[BAr₄]⁻[H(OEt₂)]⁺[B(C₆F₅)₄]⁻, B(C₆F₅)₃, PhNMe₂HB(C₆F₅)₄ and Ph₃CB(C₆F₅)₄.

49. The process of claim 48 wherein the activator is methylalumoxane, B(C₆F₅)3, PhNMe₂HB(C₆F₅)₄ or Ph₃CB(C₆F₅)₄.

50. The process of claim 48 wherein the trialkyl-aluminum is selected from the group consisting of trimethyl-aluminum, triethylaluminum and tri(isobutyl)aluminum.

51. The process of claim 48 wherein activation is achieved by the addition of a trialkylaluminum and B(C₆F₅)₃.

52. The process of claim 48 wherein activation is achieved by the addition of a trialkylaluminum and PhNMe₂HB(C₆F₅)₄.

53. The process of claim 1 wherein the soluble metal precursor has the general formula MRₙ, wherein M is a metal atom selected from the group consisting of a transition metal, a lanthanide metal or a main group metal, R is one or more solublizing ligands selected from the group consisting of mesityl and 2,4-petanedionate, and n ranges from about 2 to about 3.

54. The process of claim 1 wherein one or more of the metal-ligand compositions is screened for homogeneous catalytic activity.

55. The process of claim 54 wherein one or more of the metal-ligand compositions is screened for homogeneous catalytic activity in an olefin polymerization reaction.

56. The process of claim 1 wherein the reaction product is screened for a property of interest selected from the group consisting of electrical, thermal, mechanical, morphological, optical, magnetic or chemical.

57. The process of claim 1 wherein the reaction product is screen for a property of interest by a method selected from the group consisting of infrared spectroscopy, infrared imaging, liquid chromatography, light scattering, polymer structural testing, polymer melt flow testing, polymer property testing, Fourier transform infrared spectroscopy, thin layer chromatography, solid phase staining, rapid gel permeation chromatography, nuclear magnetic resonance spectroscopy, depolarized light scattering, rapid thermal analysis, gas composition-mass spectrometry, thermal measurement, X-ray fluorescence, reactant consumption or reactant uptake, gravimetric measurement of product yield and liquid composition-mass spectrometry.

58. The process of claim 1 wherein the array comprises at least about 8 different ligands.

59. The process of claim 1 wherein the array comprises at least about 15 different ligands.

60. The process of claim 1 wherein the array comprises at least about 23 different ligands.

61. The process of claim 1 wherein the array comprises at least about 40 different ligands.

62. The process of claim 1 wherein the array comprises at least about 80 different ligands.

63. The process of claim 1 wherein the array comprises at least about 200 different ligands.

64. The process of claim 1 wherein the array comprises at least about 1000 different ligands.

65. The process of claim 1 wherein the soluble metal precursor has the formula ML, MRₙLₘ or MₚR_{n′}L_{m′}, wherein M is a metal, R is a solublizing ligand, and L is a neutral solublizing ligand.

66. The process of claim 65 wherein L is selected from the groups consisting of alkylnitrile, cyclic olefin, bicyclic olefin, cycloalkenyl, bicycloalkenyl, alkynyl, η⁶-arene, or a combination thereof.

67. The process of claim 66 wherein L is selected from the group consisting of acetonitrile, cyclooctene, norbornene, 1,5-cyclooctadiene, cycloheptatriene, cyclooctatetraene, norbornadiene, η⁶-p-cymene, or a combination thereof.

68. The process of claim 1 wherein the soluble metal precursor is a dimer, a trimer, or higher order complex.

69. The process of claim 1 wherein the soluble metal precursor is selected from the group consisting of Ti(CH₂Ph)₄, Zr(CH₂Ph)₄, Hf(CH₂Ph)₄, V(mes)₃(THF), Ta(CH₃)₃(Cl)₂, Nb(CH₃)₃(Cl)₂, Ta(NMe₂)₃(Cl)₂, Cr[(CH(SiMe₃)₂]₃, Cr(mesityl)₂(THF), Cr(mesityl)₂(THF)₃, [Fe(mesityl)₂]₂, [Co(mesityl)₂]₂, Co(mesityl)₃Li(THF)₄, [Mn(mesityl)₂]₃, Cr(mesityl)₃, Sc(CH(SiMe₃)₂)₃, Y(CH(SiMe₃)₂)₃, Ln(CH(SiMe₃)₂)₃, Sc(0(2,6-(tBu)₂C₆H₃))₃, Y(0(2,6- (tBu)₂C₆H₃))₃, Ln(O(2,6-(tBu)₂C₆H₃))₃, Sc(0(2,6-(tBu)₂4-Me-C₆H₃))₃, Y(O(2,6-(tBu)₂4-Me-C₆H₃))₃, Ln(0(2,6-(tBu)₂4-Me-C₆H₃))₃, Sc(N(SiMe₃)₂)₃, Y(N(SiMe₃)₂)₃, Ln(N(SiMe₃)₂)₃, Ni(acac)₂, Pd(acac)₂, Co(acac)₃, Fe(acac)₃, Fe(acac)₂, Mn(acac)₂, Cr(acac)₂, Cr(acac)₃, V(acac)₃, V(O) (acac)₃, Ni(TFA)₂, Fe(TFA)_{2,} Fe(TFA)₃, Co(TFA)₂, Mn(TFA)₂, [Cr(TFA)₂]₂, Cr(TFA)₃, V(TFA)₃, CrCl₃(THF)₃, VCl₃(THF)₃, (COD)PdMeCl, [(cyclooctene)PdMeCl]₂, (COD)PdMeOTf, [(allyl)PdCl]₂, ((CH₃)₃CN)₂PdCl₂, [(allyl)NiCl]₂, [(CH₃O₂CC₃H₄)NiBr] ₂, [(allyl)NiTFA]₂, (p-cymene)-Ru(TFA)₂(CH₃CN), (p-cymene)Ru(mesityl)(TFA), (PPh₃)₄RuH₂, (PPh₃)₂Ni(Ph)Cl, (PPh₃)₄Ni, (COD)₂Ni, (py)₂Ni(CH₂SiMe₃)₂, Fe(C(SiMe₃)₃)₂, Co(C(SiMe₃)₃)₂, Mn(C(SiMe₃)₃)₂, Ti(CH₂CMe₃)₄, Zr(CH₂CMe₃)₄, Hf(CH₂CMe₃)₄, TiCl₄, Ti(NMe₂)₄, Zr(NMe₂)₄, Hf (NMe₂)₄, Zr(NEt₂)₄, Ti(NMe₂)₂Cl₂, Zr(N(SiMe₃)₂)₂Cl₂, Hf(N(SiMe₃)₂)₂Cl₂, Zr(TFA)₄, Hf(TFA)₄, Ti(TFA)₂Cl₂, TiCl₃(THF)₃, V(CH(SiMe₃)₂)₃(THF), VO-2,6-iPr-C₆H₃)₄Li(THF), Ta(NMe₂)₅, (TMEDA)NiMe₂, (TMEDA)PdMe₂, Ta(CH₂CMe₃)₂Cl₃, TaPh₅, Co(Ph₃P)₃CH₃, [Co(Ph₃P)₃H]₂N₂, and [Ni(PCY₃)₂]₂N₂, or a combination thereof, wherein Ln is selected from the group consisting of La, Ce, Pr, Nd, Sm, Eu, Gd, Tb, Dy, Ho, Er, Tm, Yb, Lu or a combination thereof.

70. The process of claim 1 further comprising delivering a metal-binding ligand which is not bound to a solid support to the plurality of discrete reaction vessels to form a metal-ligand composition, the ligand being combined with a deprotonating agent.

71. The process of claim 70 wherein the deprotonating agent is selected from the group consisting of LiN(SiMe₃)₂, NaN(SiMe₃)₂, KN(SiMe₃)₂, BuLi, or KCH₂Ph.

72. The process of claim 1 wherein the soluble metal precursor is not bound to a solid support.

73. A process for preparing and screening an array of metal-ligand compositions comprising:
preparing an array of reaction mixtures in a series of discrete reaction vessels contained by or within an integrated structure, each reaction mixture of the array comprising, in a non-protic medium, a reactant for a chemical reaction of interest and a metal-ligand composition for catalyzing a chemical reaction in which the reactant participates wherein different reaction vessels of the array contain different metal-ligand compositions and said preparing comprises delivering a metal-binding ligand not bound to a solid material and a dissolved, soluble metal precursor to each of the reaction vessels of the array;
subjecting the reaction mixture to conditions conducive to the formation of a reaction product; and
screening the array for reaction product.

74. The process of claim 73 wherein the metal-ligand compositions comprise a cyclic structure having at least 5 ring members, one member being the metal of the soluble metal precursor.

75. The process of claim 73 wherein the conditions conducive to the formation of a reaction product are selected from those suitable for carbonylation, hydroformylation, hydroxycarbonylation, hydrocarbonylation, hydroesterification, hydrogenation, transfer hydrogenation, hydrosilylation, hydroboration, hydroamination, epoxidation, aziridination, reductive amination, aryl amination, polymerization, oligomerization, C-H activation-insertion, C-H activation-insertion, C-H activation-substitution, C-halogen activation, C-halogen activation-insertion, C-halogen activation-substitution, C-C coupling, C-N coupling, cyclopropanation, alkene metathesis and alkyne metathesis.

76. The process of claim 73 wherein the metal-ligand compositions comprise a cyclic structure having at least 5 ring members, one member being the metal of the soluble metal precursor.

77. The process of claim 73 wherein the metal-binding ligand is delivered to the series of discrete reaction vessels after the soluble metal precursor.

78. The process of claim 73 wherein the soluble metal precursor has a formula MRₙ, wherein M is a transition metal, a lanthanide metal, an actinide metal, an alkali metal, an alkaline earth metal or a main group metal, R is a solublizing ligand, and n ranges from about 1 to about 8.

79. The process of claim 73 wherein the soluble metal precursor has a formula MₚR_{n′}L_{m′}, wherein M is a transition metal, a lanthanide metal, an actinide metal, an alkali metal, an alkaline earth metal or a main group metal, R is a solublizing ligand, L is a neutral solublizing ligand, p is greater than 1, n′ and m′ are each independently a number greater than 0, provided the sum of n′+m″ is at least 2.

80. The process of claim 73 wherein the soluble metal precursor comprieses a metal selected from Sc, Ti, V, Cr, Mn, Fe, Co, Ni, Cu, Zn, Y, Zr, Nb, Mo, Ru, Rh, Pd. Ag, Cd, La, Hf, Ta, W, Re, Os, Ir, Pt, Au, Hg, Ce, Pr, Nd, Sm, Eu, Gd, Tb, Dy, Ho, Er, Tm, Yb, Lu, U, Th, B, Al, Ga, Tl, In, Li, Na, K, Rb, Cs, Be, Mg, Ca, Sr, Ba, Ge, Sn, Pb, Sb, Bi or a combination thereof.

81. The process of claim 73 wherein the soluble metal precursor comprises one or more solublizing ligands independently selected from the group consisting of halogen, substituted or unsubstituted alkyl, substituted or unsubstituted cycloalkyl, substituted or unsubstituted cycloalkenyl, substituted or unsubstituted heteroalkyl, substituted or unsubstituted heterocycloalkyl, substituted or unsubstituted aryl, substituted or unsubstituted heteroaryl, alkoxy, aryloxy, hydroxy, boryl, silyl, hydride, thio, oxo, imido, sulfido, seleno, phosphino, amino, carboxylate, 1,3-dionate, oxalate, carbonate, nitrate, sulfate, perchlorate, sulfonate, phosphonate and combinations thereof.

82. The process of claim 73 wherein the soluble metal precursor comprises a solublizing ligand R and/or a neutral solublizing ligand L, and further wherein one or more of the metal-ligand compositions is formed without displacing one or more of the solublizing ligands and/or neutral solublizing ligands.

83. The process of claim 73 wherein the soluble metal precursor comprises a solublizing ligand R and/or a neutral solublizing ligand L, and further wherein formation of one or more of the metal-ligand compositions is accompanied by the displacement of one or more of the solublizing ligands and/or neutral solublizing ligands, said displaced ligand(s) not interfering with or significantly inhibiting the reaction.

84. The process of claim 83 wherein, prior to polymerization reaction, one or more of the metal-ligand compositions is treated to convert the displaced solublizing ligand, R, and/or the displaced neutral solublizing ligand, 5 L, to a form which does not interfere or significantly inhibit the reaction.

85. The process of claim 73 wherein each metal-binding ligand has a coordination number (CN) independently selected from the group consisting of 1, 2, 3 and 4.

86. The process of claim 85 wherein each metal-binding ligand has a charge independently selected from the group consisting of 0, −1, −2 and −3.

87. The process of claim 73 wherein each metal-binding ligand is independently selected from the group consisting of derivatives of the following:

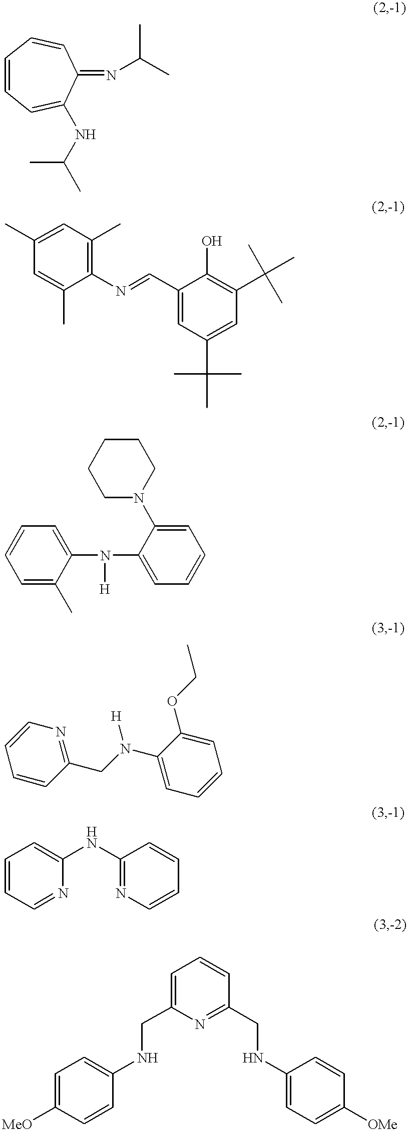

88. The process of claim 73 wherein the soluble metal precursor comprises more than one solublizing ligand, each being the same.

89. The process of claim 88 wherein the solublizing ligand is selected from the group consisting of halo, hydride, substituted or unsubstituted alkyl, substituted or unsubstituted cyclocalkyl, substituted or unsubstituted aryl, substituted or unsubstituted heteroalkyl, alkoxy, aryloxy, dionate, carboxylate and amino.
